(19) United States
(12) Patent Application Publication
(10) Pub. No.: US 2009/0156644 A1
(43) Pub. Date: Jun. 18, 2009

(54) Use of thiazole derivatives and analogues in the treatment of cancer

(76) Inventors: Jacob Westman, Jarlasa (SE); Guido Kurz, Madrid (ES); Bjorn Eriksson, Umea (SE); Christian Hedberg, Dortmund (DE)

Correspondence Address: MORGAN LEWIS & BOCKIUS LLP, 1111 PENNSYLVANIA AVENUE NW, WASHINGTON, DC, 20004, US

(21) Appl. No.: 11/989,029

(22) PCT Filed: Jul. 21, 2006
(86) PCT No.: PCT/GB2006/002730

§ 371 Date: Dec. 2, 2008

Related U.S. Application Data
(60) Provisional application No. 60/595,620, filed on Jul. 21, 2005.
(60) Provisional application No. 60/744,422, filed on Apr. 7, 2006.

(30) Foreign Application Priority Data
Jul. 21, 2005 (SE) 0501721-5

(51) Int. Cl. A61K 31/426; C07D 277/14; A61K 31/4439; C07D 417/02

(52) U.S. Cl. 514/342; 548/182; 546/269.7; 514/369

(57) ABSTRACT

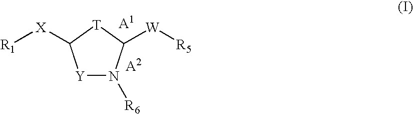

There is provided a use of a compound of formula (I), wherein X, Y, T, W, A₁, A₂R₁, R₅ and R₆ have meanings given in the description for the manufacture of a medicament for the treatment of cancer.

### FIELD OF THE INVENTION

This invention relates to a novel pharmaceutical use of certain compounds, some of which compounds are novel and/or are not known for use as pharmaceuticals. In particular, this invention relates to the use of such compounds in the treatment of cancer.

### BACKGROUND AND PRIOR ART

Elevated plasma free fatty acids (FFAs) stimulate pancreatic β-cells and is one cause of hyperinsulinemia.

Excess adiposity is associated to different degrees with an increased risk of developing cancers, such as colorectal adenomas, breast cancer (postmenopausal), endometrial cancer, kidney cancer, oesophageal adenocarcinoma, ovarian cancer, prostate cancer, pancreatic cancer, gallbladder cancer, liver cancer and cervical cancer (Calle and Kaaks (2004), Nature Reviews Cancer, 4, 579-591).

Recent studies suggest that hyperinsulinemia is correlated among other things to the incidence of colon and lethal breast and prostate cancer.

In prostate cancer, hyperinsulinemia has been shown to be prospective risk factor for death and data support that the insulin level could be used as a marker of prostate cancer prognosis (Hammarsten and Högstedt (2005) European Journal of Cancer, 41, 2887).

Several mechanisms may link hyperinsulinemia to the incidence and outcome of breast cancer. Firstly, chronic hyperinsulinemia results in increased production of ovarian testosterone and oestrogen and inhibition of hepatic production of sex hormone binding globulin, a sex-hormonal profile that is associated with breast cancer. Secondly, hyperinsulinemia suppresses hepatic production of insulin-like growth factor binding protein-1 (IGFBP-1), and thus increases circulating levels of IGF-1, which has potent mitogenic effect on breast tissue. Thirdly, insulin itself may have a direct mitogenic effect on breast cancer cells.

The study by Hardy et al ((2005), J. Biol. Chem. 280, 13285) shows that FFAs directly stimulate the growth of breast cancer cells in a GPR40 dependent manner. Moreover, expression studies performed on tumor tissue isolated from 120 breast cancer patient shows a frequent expression of GPR40 emphasizing the clinical relevance of the findings of Hardy (see, for example, Ma et al, Cancer Cell (2004) 6, 445).

Another expression study on clinical material from colon cancer patients suggests that similar mechanisms could be relevant also in these malignancies (see http://www.ncbi.nlm.nih.gov/projects/geo/gds/gds_browse.cgi?gds=1263).

U.S. Pat. No. 1,293,741 discloses inter alia thiazolidinones. However, there is no mention of the use of the compounds disclosed therein in the treatment of cancer.

U.S. Pat. No. 4,103,018 and U.S. Pat. No. 4,665,083 disclose inter alia thiazolidinones. However, there is no mention or suggestion of the compounds disclosed in those documents in the treatment of cancer.

WO 2005/051890 discloses inter alia thiazolidinones (which are ultimately substituted with a cyclopropyl group) that may be useful in the treatment of diabetes. However, there is no mention or suggestion in this document of the use of the compounds in the treatment of cancer.

EP 1 535 915 discloses various furan and thiophene-based compounds. Cancer is mentioned as one of numerous indications.

EP 1 559 422 discloses a huge range of compounds for use in the treatment of inter alia cancer. However, this document does not appear to relate to thiazolidinones.

International patent applications WO 2005/075471 and WO 2005/116002 disclose inter alia thiazolidinones and oxazolidinones as 11-β-hydroxysteroid dehydrogenase type 1 inhibitors. There is no mention or suggestion of the use of the disclosed compounds for the treatment of cancer.

International patent application WO 2006/040050 discloses certain quinazolinylmethylene thiazolinones as CDK1 inhibitors. Similarly, US patent application US 2006/0004045 discloses quinolinylmethylene thiazolinones.

We have now surprisingly found compounds that are able to antagonize the stimulatory effect of FFAs on cell proliferation when tested in an assay using a human breast cancer cell line (MDA-MB-231). The compounds may thus possess a surprisingly beneficial inhibitory effect on the ability of tumors of this type, and of cancers generally, to survive.

### DISCLOSURE OF THE INVENTION

According to the invention there is provided a use of a compound of formula I,

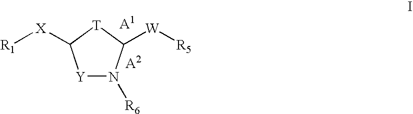

wherein
X represents —[C(R₈)(R₉)]ₙ—;
n represents 0, 1, 2 or 3;
Y represents —C(O)—, —S(O)₂— or ═C(R₁₀)—;
T represents —S— or —O—;
W represents —NR₇—, —CR₇R₇—, —NR₇C(O)—, —NR₇S(O)₂—, —NR₇C(O)NR₇—, —NR₇C(O)O— or a bond;
one of A₁ or A₂ represents a double bond and the other represents a single bond;
when A₁ represents a single bond, A₂ is a double bond and R₆ is absent;
when A₂ represents a single bond, A₁ is a double bond and, if present, one R₇ (which is attached a to the requisite ring of the compound of formula I) is absent;
R₁ represents —C(O)NR₃R₂, —NR₃R₂, —C(O)OR₂, —NR₄C(O)NR₃R₂, —NR₄C(O)OR₂, —OC(O)NR₃R₂, —NR₄C(O)R₂, —OC(O)R₂, —OR₂, —SR₂, H, alkyl, cycloalkyl, heterocyclyl, benzyl, aryl or heteroaryl (which latter six groups are optionally substituted by one or more groups selected from B¹, B², B³, B⁴, B⁵ and B⁶, respectively);
R₂ and R₅ independently represent, on each occasion when used herein, hydrogen, alkyl, cycloalkyl, heterocyclyl, benzyl, aryl or heteroaryl (which latter six groups are optionally substituted by one or more groups selected from B⁷, B⁸, B⁹, B¹⁰, B¹¹ and B¹², respectively);
R₃, R₄, R₆ and R₇ independently represent, on each occasion when used herein, hydrogen, alkyl, cycloalkyl, aryl or benzyl (which latter four groups are optionally substituted by one or more groups selected from B¹³, B¹⁴, B¹⁵ and B¹⁶, respectively), or heterocyclyl or heteroaryl (which latter two groups are optionally substituted by one or more groups selected from B¹⁴ and B¹⁵, respectively);
R₈ and R₉ are independently selected from hydrogen, alkyl and aryl (which latter two groups are optionally substituted by B^{16a} and B^{16b}, respectively);
R₁₀ represents hydrogen, alkyl or aryl (which latter two groups are optionally substituted by one or more groups selected from B¹⁷ and B¹⁸, respectively);
B¹ to B¹⁸ independently represent cyano, —NO₂, halo, —OR₁₁, —NR₁₂R₁₃, —SR₁₄, —Si(R₁₅)₃, —C(O)OR₁₆, —C(O)NR₁₆ₐR_{16b}, —S(O)₂NR_{16c}R_{16d}, aryl or heteroaryl (which aryl and heteroaryl groups are themselves optionally and independently substituted by one or more groups selected from halo and R₁₇); or, alternatively,
B⁴, B⁵, B⁶, B¹⁰, B¹¹, B¹², B¹⁵, B¹⁶, B^{16b} or B¹⁸ independently represent R₁₇;
R₁₁, R₁₂, R₁₃, R₁₄, R₁₆, R₁₆ₐ, R_{16b}, R_{16c} and R_{16d} independently represent H or R₁₇; and
R₁₅ and R₁₇ independently represent, on each occasion when used herein, C₁₋₆ alkyl optionally substituted by one or more halo atoms,
or a pharmaceutically-acceptable salt or solvate, or a pharmaceutically functional derivative thereof,
for the manufacture of a medicament for the treatment of cancer.

Pharmaceutically-acceptable salts that may be mentioned include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Examples of pharmaceutically acceptable addition salts include those derived from mineral acids, such as hydrochloric, hydrobromic, phosphoric, metaphosphoric, nitric and sulphuric acids, and organic acids, such as tartaric, acetic, citric, malic, lactic, fumaric, benzoic, glycolic, gluconic, succinic, and arylsulphonic acids.

“Pharmaceutically functional derivatives” of compounds of formula I as defined herein includes ester derivatives and/or derivatives that have, or provide for, the same biological function and/or activity as any relevant compound. Thus, for the purposes of this invention, the term also includes prodrugs of compounds of formula I.

The term “prodrug” of a relevant compound of formula I includes any compound that, following oral or parenteral administration, is metabolised in vivo to form that compound in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration.

Prodrugs of compounds of formula I may be prepared by modifying functional groups present on the compound in such a way that the modifications are cleaved, in vivo when such prodrug is administered to a mammalian subject. The modifications typically are achieved by synthesizing the parent compound with a prodrug substituent. Prodrugs include compounds of formula I wherein a hydroxyl, amino, sulfhydryl, carboxy or carbonyl group in a compound of formula I is bonded to any group that may be cleaved in vivo to regenerate the free hydroxyl, amino, or sulfhydryl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elesevier, N.Y.-Oxford (1985).

Compounds of formula I, as well as pharmaceutically-acceptable salts, solvates and pharmaceutically functional derivatives of such compounds are, for the sake of brevity, hereinafter referred to together as the “compounds of formula I”.

Compounds of formula I may contain double bonds and may thus exist as E (entgegen) and Z (zusammen) geometric isomers about each individual double bond. All such isomers and mixtures thereof are included within the scope of the invention.

Compounds of formula I may exist as regioisomers and may also exhibit tautomerism. All tautomeric forms and mixtures thereof are included within the scope of the invention. Specifically, tautomers exist when R⁶ represents H. Such compounds have different point of attachments of R⁶ accompanied by one or more double bond shifts.

Compounds of formula I may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise stated, the term “alkyl” refers to an unbranched or branched, cyclic, saturated or unsaturated (so forming, for example, an alkenyl or alkynyl) hydrocarbyl radical, which may be substituted or unsubstituted (with, for example, B¹, B², B⁷, B⁸, B¹³, B¹⁴, B^{16a} or B¹⁷). Where the term “alkyl” refers to an acyclic group, it is preferably C₁₋₁₀ alkyl and, more preferably, C₁₋₆ alkyl (such as ethyl, propyl, (e.g. n-propyl or isopropyl), butyl (e.g. branched or unbranched butyl), pentyl or, more preferably, methyl). Where the term “alkyl” is a cyclic group (which may be where the group “cycloalkyl” is specified), it is preferably C₃₋₁₂ cycloalkyl and, more preferably, C₅₋₁₀ (e.g. C₅₋₇) cycloalkyl.

When used herein, alkylene refers to C₁₋₁₀ (e.g. C₁₋₆) alkylene and, preferably C₁₋₃ alkylene, such as pentylene, butylene (branched or unbranched), preferably, propylene (n-propylene or isopropylene), ethylene or, more preferably, methylene (i.e. —CH₂—).

The term “halogen”, when used herein, includes fluorine, chlorine, bromine and iodine.

Heterocyclyl groups that may be mentioned include non-aromatic monocyclic heterocyclyl groups in which one or more (e.g. one to four) of the atoms in the ring system is other than carbon (i.e. a heteroatom, which heteroatom is preferably selected from N, O and S), and in which the total number of atoms in the ring system is between three and twelve (e.g. between five and ten). Further, such heterocycloalkyl groups may be saturated or unsaturated containing one or more double and/or triple bonds, forming for example a C_{2-q} heterocycloalkenyl (where q is the upper limit of the range) or a C_{3-q} heterocycloalkynyl group. C_{2-q} heterocycloalkyl groups that may be mentioned include 7-azabicyclo[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo[3.2.1]octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form. Preferred heterocyclyl groups include cyclic amino groups such as pyrrolidinyl, piperidyl, piperazinyl, morpholinyl or a cyclic ether such as tetrahydrofuranyl, monosaccharide.

The term “aryl” when used herein includes C₆₋₁₄ (such as C₆₋₁₃ (e.g. C₆₋₁₀)) aryl groups. Such groups may be monocyclic, bicyclic or tricyclic and have between 6 and 14 ring carbon atoms, in which at least one ring is aromatic. The point of attachment of aryl groups may be via any atom of the ring system. However, when aryl groups are bicyclic or tricyclic, they are linked to the rest of the molecule via an aromatic ring. C₆₋₁₄ aryl groups include phenyl, naphthyl and the like, such as 1,2,3,4-tetrahydronaphthyl, indanyl, indenyl and fluorenyl. Most preferred aryl groups include phenyl.

The term “heteroaryl” when used herein refers to an aromatic group containing one or more heteroatom(s) (e.g. one to four heteroatoms) preferably selected from N, O and S (so forming, for example, a mono-, bi-, or tricyclic heteroaromatic group). Heteroaryl groups include those which have between 5 and 14 (e.g. 10) members and may be monocyclic, bicyclic or tricyclic, provided that at least one of the rings is aromatic. However, when heteroaryl groups are bicyclic or tricyclic, they are linked to the rest of the molecule via an aromatic ring. Heterocyclic groups that may be mentioned include benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-α]pyridyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. Particularly preferred heteroaryl groups include pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, thiophenetyl, pyranyl, carbazolyl, acridinyl, quinolinyl, benzoimidazolyl, benzthiazolyl, purinyl, cinnolinyl and pterdinyl.

For the avoidance of doubt, in cases in which the identity of two or more substituents in a compound of formula I may be the same, the actual identities of the respective substituents are not in any way interdependent. For example, in the situation in which R¹ and R² are both aryl groups substituted by one or more C₁₋₆ alkyl groups, the alkyl groups in question may be the same or different.

For the avoidance of doubt, when a term such as “B¹ to B¹⁸” is employed herein, this will be understood by the skilled person to mean B¹, B², B³, B⁴, B⁵, B⁶, B⁷, B⁸, B⁹, B¹⁰, B¹¹, B¹², B¹³, B¹⁴, B¹⁵, B¹⁶, B^{16a}, B^{16b}, B¹⁷ and B¹⁸ inclusively.

For the avoidance of doubt, when the group ‘benzyl’ is substituted, then the substituents are preferably on the phenyl ring of the benzyl group, rather than on the methylene (—CH₂—) group.

For the avoidance of doubt, when Y represents ═C(R¹⁰)—, this refers to the following compound of formula Ia

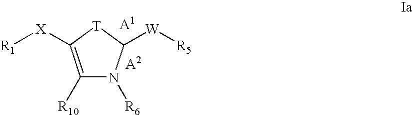

Compounds of formula I that may be mentioned include those in which:

Y preferably represents —C(O)—;
R₁ represents —C(O)NR₃R₂, —NR₃R₂, —C(O)OR₂, —NR₄C(O)NR₃R₂, —NR₄C(O)OR₂, —OC(O)NR₃R₂, —NR₄C(O)R₂, —OC(O)R₂, —OR₂, —SR₂, H, alkyl, haloalkyl cycloalkyl, heterocyclyl, benzyl, aryl or heteroaryl;
R₂ and R₅ independently represent, on each occasion when used herein, hydrogen, alkyl, haloalkyl, cycloalkyl, heterocyclyl, benzyl, aryl or heteroaryl;
R₃, R₄, R₆ and R₇ independently represent, on each occasion when used herein, aryl or, more particularly, hydrogen, alkyl, haloalkyl, cycloalkyl or benzyl;
R₈ and R₉ are independently selected from hydrogen, alkyl and aryl;
R₁₀ represents hydrogen, alkyl, haloalkyl or aryl.

Further compounds of formula I that may be mentioned include those in which:

B¹ to B¹⁸ independently represent halo, —OR₁₁, —NR₁₂R₁₃, —SR₁₄, —Si(R₁₅)₃, —C(O)OR₁₆ or aryl (which aryl group is itself optionally substituted by one or more groups selected from halo or R₁₇, or is preferably unsubstituted);
R₁₁, R₁₂, R₁₃, R₁₄ and R₁₆ independently represent R₁₇ or, more preferably, H.

B¹ to B¹⁸ may alternatively independently represent functional groups such as hydroxyl, amine, sulfide, silyl, carboxylic acid, halogen, aryl, etc.

Further compounds of formula I that may be mentioned include those in which:

Y represents —C(O)—;
T represents —S—;
n represents 1;
W represents —N—;
A₂ represents a single bond and A₁ is a double bond; and/or R₆ represents H;
R₁ and R₅ independently represent aryl or heteroaryl.

Further compounds of formula I that may be mentioned include those in which:

X is alkylene or a bond (i.e. when n represents 0);
T represents —S—;
Y represents ═C(H)— or, preferably —C(O)—;
W represents —NR₇—;
A₁, A₂, R₁, R₂ and R₅ are as hereinbefore defined; and/or
R₃, R₄ and R₆ independently represent hydrogen, alkyl (e.g. optionally substituted by one or more groups selected from B¹³), haloalkyl, cycloalkyl (e.g. optionally substituted by one or more groups selected from B¹⁴) or benzyl (e.g. optionally substituted by one or more groups selected from B¹⁶).

More preferred compounds of formula I include those in which:

X represents —CH₂—;
Y represents —C(O)—;
R₁ and R₂ independently represent aryl (e.g. phenyl) as hereinbefore defined (i.e. R₁ represents aryl optionally substituted by one or more B⁵ groups and R₂ represents aryl optionally substituted by one or more B¹¹ groups);
when R₁ and/or R₂ represent phenyl, it/they is/are substituted para relative to the point of attachment of the R₁ or R₂ group to X;
B⁵ and B¹¹ independently represent halo; and/or
R₅ represents heteroaryl (e.g. pyridyl).

More preferred compounds of formula I include those in which:

R₁ represents —C(O)NHR₂;
R₂ represents aryl (e.g. phenyl);
when R₂ represents phenyl, it is substituted (i.e. with a B¹¹ substituent) at the para position (relative to the point of attachment of the R₂ group to the remainder of the compound of formula I); and/or
B¹¹ represents C₁-C₆ alkyl.

In another preferred embodiment of the present invention:

### R₁ is —NHR₂;

R₂ is aryl (e.g. phenyl);
when R₂ represents phenyl, it is substituted (i.e. with a B¹¹ substituent) at the para position;
B¹¹ represents C₁-C₆ alkyl;
Y represents ═C(H)—;
R₅ represents aryl (e.g. phenyl); and/or
when R₅ represents phenyl, it is either unsubstituted or substituted with a halogen (i.e. B¹¹ represents halo).

In a still another preferred embodiment of the present invention:

R₅ represents aryl (e.g. phenyl);
when R₅ represents phenyl, it is substituted (i.e. with a B¹¹ substituent) at the para position; and/or
B¹¹ represents R₁₇;
R₁₇ represents C₁₋₆ alkyl preferably substituted by one or more halo atoms (so forming a haloalkyl group).

In a still another preferred embodiment of the present invention;

Y represents ═C(H)—;
R₅ represents aryl (e.g. phenyl);
when R₅ represents phenyl, it is substituted (i.e. with a B¹¹ substituent) at the para position; B¹¹ represents halo or R₁₇; and/or
R₁₇ represents C₁₋₆ alkyl preferably substituted by one or more halo atoms (so forming a haloalkyl group).

In a still another preferred embodiment of the present invention:

X represents a single bond (i.e. n represents 0);

### R₁ is —C(O)NHR₂;

R₂ is aryl (e.g. phenyl);
when R₂ represents phenyl, it is substituted with B¹¹;
B¹¹ represents R₁₇; and/or
R₁₇ represents C₁-C₆ alkyl.

Preferred compounds of formula I include those in which:

T represents —S—;
Y represents ═C(R₁₀)—, preferably, —S(O)₂— or, more preferably, —C(O)—;
R₁₀ represents H or, more preferably, alkyl (e.g. methyl or trifluoromethyl);
W represents —CR₇R₇—, a bond, or, more preferably, —NR₇—, —NR₇C(O)—, —NR₇C(O)O—, —NR₇C(O)NR₇— or —NR₇S(O)₂—;
R₅ represents optionally substituted (i.e. by B⁷) alkyl (such as C₁₋₃ alkyl, e.g. propylene or. preferably, isopropyl or methyl; so forming, for example, a benzyl group), cycloalkyl (e.g. cyclohexyl) or, more preferably represents optionally substituted (i.e. by B¹¹) aryl (e.g. phenyl) or optionally substituted (i.e. by B¹²) heteroaryl (e.g. 2-pyridyl);
n represents 3 or 0 or, more preferably, 1 or 2;
R₈ and R₉ independently represent C₁₋₃ (e.g. C₁₋₂) alkyl (e.g. methyl) or, more preferably, H;
R₁, represents (e.g. when n represents 1) alkyl or, more preferably —NR₃R₂, —OR₂, —SR₂, —NR₄C(O)R₂, —NR₄C(O)NR₃R₂, —NR₄C(O)OR₂, particularly —C(O)NR₃R₂, —C(O)OR₂, more particularly, optionally substituted (i.e. by B⁶) heteroaryl (e.g. furanyl, such as furan-2-yl or thienyl, such as thien-2-yl) or, especially, optionally substituted (i.e. by B⁵) aryl (e.g. phenyl);
when n represents 0, then R₁ preferably represents alkyl, such as C₁₋₃ alkyl (e.g. propyl or methyl), which group is saturated or unsaturated (e.g. contains one or two double bonds, one of which is, for example, directly attached to the requisite 5-membered ring of formula I) so forming, for example, a methenyl (i.e. a ═CH₂) or a propdienyl (i.e. ═CH—CH═CH—) group, and which group is unsubstituted or, preferably, substituted (e.g. at the terminal position) by one or more (e.g. one) B¹ group (so forming, for example, a —C(OH)(H)— or, preferably, a benzyl group);
R₄ represents C₁₋₃ (e.g. C₁₋₂) alkyl (e.g. methyl) or H;
R₃ represents C₁₋₃ (e.g. C₁₋₂) alkyl (e.g. methyl) or, preferably, H;
R₂ represents optionally substituted (i.e. by B⁷) alkyl (such as C₁₋₃ alkyl, e.g. ethyl or, preferably, methyl; so forming, for example, a benzyl group) or, preferably, optionally substituted (i.e. by B¹¹) aryl (e.g. phenyl) or (e.g. when R₁ represents —C(O)OR₂) H;
when W represents —NR₇— and R₇ is absent, then R₆ represents alkyl such as C₁₋₆ (e.g. C₁₋₃) alkyl (e.g. methyl) or aryl (e.g. phenyl), both of which may be substituted by one or more of B¹³ or B¹⁵, respectively, or are more preferably unsubstituted, or, more preferably R₆ represents H;
when W represents —NR₇— and R₆ is absent, then R₇ represents C₁₋₃ (e.g. C₁₋₂) alkyl (e.g. methyl), aryl (e.g. phenyl) or benzyl, all of which may be substituted by one or more B¹³, B¹⁵ and B¹⁶, respectively, or, are more preferably unsubstituted;
when W represents —CR₇R₇—, then A₂ represents a double bond;
when W represents —CR₇R₇—, then each R₇ independently represents, at each occurrence, C₁₋₃ (e.g. C₁₋₂) alkyl or H;
B¹ to B¹⁸ (and, in particular, B⁵, B⁶, B¹¹ and B¹²) independently represent cyano, NO₂, halo (e.g. chloro, fluoro or bromo), —OR₁₁, —C(O)OR₁₆, —C(O)NR₁₆ₐR_{16b} or —S(O)₂NR_{16c}R_{16d}; and/or
B⁴ to B⁶, B¹⁰ to B¹², B¹⁵, B¹⁶ and B¹⁸ (and, in particular, B⁵, B¹¹ and B¹²) represents R₁₇; and/or
B¹ to B¹⁸ (and, in particular, B¹ and B⁷) independently represent heteroaryl (e.g. furanyl, such as furan-2-yl or thienyl, such as thien-2-yl) or, preferably, aryl (e.g. phenyl), both of which may be substituted by one or more groups selected from halo (e.g. fluoro) or R₁₇;
R₁₁ represents C₁₋₃ (e.g. C₁₋₂) alkyl (e.g. methyl or ethyl) or H;
R₁₆ represents H or C₁₋₃ (e.g. C₁₋₂) alkyl (e.g. ethyl);
R₁₆ₐ, R_{16b}, R_{16c} and R_{16d} independently represent C₁₋₂ alkyl or, more preferably, H;
R₁₇ represents C₁₋₄ (e.g. C₁₋₃) alkyl (e.g. methyl or isopropyl) optionally substituted by one or more halo (e.g. fluoro) atoms (so forming, for example, a trifluoromethyl group).

It preferred that:

R₁₀ does not represent H;
when Y represents ═C(R₁₀)—, W does not represent —N(R₇)C(O)—;
n represents 1, 2 or 3;
R₃, R₄, R₆ and R₇ independently represent, on each occasion when used herein, hydrogen, alkyl, cycloalkyl, aryl or benzyl (which latter four groups are optionally substituted by one or more groups selected from B¹³, B¹⁴, B¹⁵ and B¹⁶, respectively;
R₁ does not represent H or alkyl as hereinbefore defined;
R₅ does not represent H.

Preferred compounds of formula I include those in which:

when X represents a single bond (i.e. n represents 0) and R₁ represents an optionally substituted alkyl group, then it is preferably saturated;
when X does not represent a single bond (i.e. n does not represent 0), then R₁ does not represent —NR₃R₂, —OR₂, —SR₃, —NR₄C(O)R₂, —NR₄C(O)NR₃R₂ or —NR₄C(O)OR₂;
when X represents —CH₂—, R₁ represents optionally substituted aryl, W represents —NR₇—, then:
(i) R₅ does not represent alkyl or cycloalkyl; or
(ii) R₅ does not represent hydrogen;
when X represents a single bond (i.e. n represents 0) and R₅ represents optionally substituted aryl, then R₁ does not represent an optionally substituted alkyl group or hydrogen;
when X represents —CH₂— and R₅ represents optionally substituted aryl, then R₁ does not represent —C(O)NR₃R₂;
when X represents —CH₂— and R₅ represents optionally substituted alkyl or aryl, then R₁ does not represent —C(O)NR₃R₂.

Some compounds of formula I are novel per se. In this respect, there is further provided a compound of formula I as hereinbefore defined but in which Y represents —S(O)₂—, provided that when T represents —S—, W represents —NR₇— and:
(a) A₁ represents a double bond, n represents 0 and R₁ represents phenyl, then (i) R₅ does not represent phenyl when R₆ represents methyl and (ii) R₆ does not represent phenyl when R₅ represent methyl; and
(b) A₂ represents a double bond, n represents 1, R₁, R₇, R₈ and R₉ all represent H, then R₅ does not represent 3-chlorobenzyl.

More preferred compounds of formula I include those of the examples described hereinafter and, in particular:
5-(4-fluorobenzyl)-2-(pyridin-2-ylimino)thiazolidin-4-one;
5-(p-methylbenzyl)-2-(4-chlorophenylimino)thiazolidin-4-one;
5-(3-(trifluoromethyl)benzyl)-2-(p-tolylimino)thiazolidin-4-one;
5-(3-(trifluoromethyl)benzyl)-2-(4-chlorophenylimino)thiazolidin-4-one;
5-(3-(trifluoromethyl)benzyl)-2-(4-isopropylphenylimino)thiazolidin-4-one;
5-(3-(trifluoromethyl)benzyl)-2-(4-methoxyphenylimino)thiazolidin-4-one;
5-(3-(trifluoromethyl)benzyl)-2-(phenylimino)thiazolidin-4-one;
2-(3,4-dichlorophenylimino)-5-(3-(trifluoromethyl)benzyl)thiazolidin-4-one;
2-(2,4-dichlorophenylimino)-5-(3-(trifluoromethyl)benzyl)thiazolidin-4-one;
5-(3-(trifluoromethyl)benzyl)-2-(p-tolylimino)-3-methylthiazolidin-4-one;
N-(5-(3-(trifluoromethyl)benzyl)-4-oxothiazolidin-2-ylidene)-4-chlorobenzamide;
5-(3-(trifluoromethyl)benzyl)-2-(4-chlorophenyl)sulfonyliminothiazolidin-4-one;
phenyl 5-(3-(trifluoromethyl)benzyl)-4-oxothiazolidin-2-ylidenecarbamate;
5-(4-methoxyphenethyl)-2-(p-tolylimino)thiazolidin-4-one;
5-(4-methoxyphenethyl)-2-(phenylimino)thiazolidin-4-one; and
2-(p-tolylimino)-5-phenethylthiazolidin-4-one.

Particularly preferred compounds of formula I include:
5-(4-fluorobenzyl)-2-(pyridin-2-ylimino)thiazolidin-4-one;
5-(3-(trifluoromethyl)benzyl)-2-(4-chlorophenylimino)thiazolidin-4-one;
5-(3-(trifluoromethyl)benzyl)-2-(p-tolylimino)thiazolidin-4-one
5-(4-methoxyphenethyl)-2-(p-tolylimino)thiazolidin-4-one;
5-(4-methoxyphenethyl)-2-(phenylimino)thiazolidin-4-one; and
2-(p-tolylimino)-5-phenethylthiazolidin-4-one.

Especially preferred compounds of formula I include 5-(3-(trifluoromethyl)benzyl)-2-(4-chlorophenylimino)thiazolidin-4-one

Compounds of formula I may be known and/or may be commercially available. Other compounds of formula I (e.g. that are not commercially available) may be prepared in accordance with techniques that are well known to those skilled in the art, for example as described hereinafter.

According to a further aspect of the invention there is provided a process for the preparation of a compound of formula I, which process comprises:

(i) for compounds of formula I in which Y represents —C(O)—, W represents —NR₇, and A₁ represents a double bond (and R₇ is therefore absent), reaction of either:
(A) a compound of formula II,

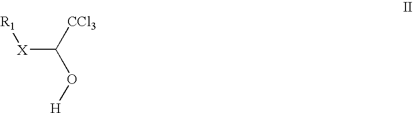

(B) a compound of formula III,

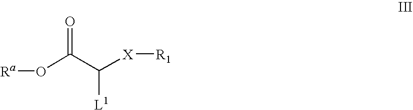

wherein R^{a} represents C₁₋₆ alkyl (e.g. ethyl; so forming an ester group), L¹ represents a suitable leaving group, such as halo (e.g. bromo or chloro) or a sulfonate group (e.g. mesylate or, preferably, tosylate); or
(C) a compound of formula IV,

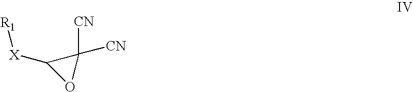

wherein, in all cases, X and R₁ are as hereinbefore defined,
   with, in each case, a compound of formula V,

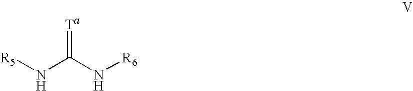

wherein T^{a} represents S or O and R₆ is as hereinbefore defined, under reaction conditions known to those skilled in the art, for example for reaction (A) above conditions such as those described in Blanchet et al, Tetrahedron Letters, 2004, 45, 4449-4452; for reaction (B) above, conditions such as those described in St. Laurent et al, Tetrahedron Letters, 2004, 45, 1907-1910; K. Arakawa et al., Chem. Pharm. Bull. 1997, 45, 1984-1993; A. Mustafa, W. Musker, A. F. A. M. Shalaby, A. H. Harhash, R. Daguer, Tetrahedron 1964, 20; 25-31; or P. Herold, A. F. Indolese, M. Studer, H. P. Jalett, U. Siegrist, H. U. Blaser, Tetrahedron 2000, 56, 6497-6499 and for reaction (C) above, conditions such as those described in Le Martchalal et al, Tetrahedron 1990, 46, 453-464;
(ii) for compounds of formula I in which Y represents —S(O)₂—, W represents —NR₇—, and A₁ represents a double bond (and R₇ is therefore absent), X represents -[R₈R₉]ₙ- in which n represents 0 and R₁ represents H, reaction of a compound of formula VI,

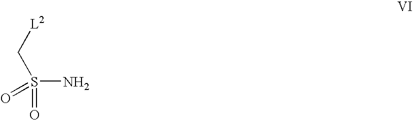

wherein L² represents a suitable leaving group, such as halo (e.g. chloro), with a compound of formula VII,

R₅—N═C=T^{a} VII

wherein T^{a} is as hereinbefore defined but is preferably S and R₅ is as hereinbefore defined under conditions known to those skilled in the art, for example such as those described in Zbirovsky and Seifert, Coll. Czech. Chem. Commun. 1977, 42, 2672-2679 or Von Zaki EI-Heweri, Franz Runge, Journal für praktische Chemie, 4, Band 16, 1962, e.g. in the presence of base (e.g. an aqueous solution of NaOH) in an appropriate solvent (e.g. acetone), for example at elevated temperature (e.g. 50°);
(iii) for compounds of formula I in which A₁ represents a double bond (and R₇ is therefore absent), X represents -[R₈R₉]ₙ- in which n represents 1, 2 or 3 and R₁ is as hereinbefore defined and, preferably, Y represents —S(O)₂— and/or W represents —NR₇, reaction of a corresponding compound of formula I in which n represents 0 and R₁, represents hydrogen, with a compound of formula VIII,

R₁ₐ-X^{a}-L³ VIII

wherein X^{a} represents -[R₈R₉]ₙ- in which n represents 1, 2 or 3 and R₁ₐ represents R₁ as hereinbefore defined, or n represent 0 and R₁ₐ represents R₁ as hereinbefore defined provided that it does not represent hydrogen, aryl or heteroaryl, and L³ represents a suitable leaving group (e.g. a halo or sulfonate group), under reaction conditions known to those skilled in the art, for example, in the presence of a suitable base (e.g. an organometallic base (e.g. an organolithium), an alkali metal base (e.g. sodium hydride) or an amide salt (e.g. (Me₃Si)₂NNa) and the like) and a suitable solvent (e.g. tetrhydrofuran, dimethylformamide, dimethlysulfoxide or the like) at room temperature or below (such as at sub-zero temperatures (e.g. −78° C.). For example, for the synthesis of compounds of formula I in which Y represents —S(O)₂— and/or W represents —NR₇, reaction conditions include those described in the journal article mentioned in respect of process step (ii) above;
(iv) for compounds of formula I in which n represents 0 and R₁ represents alkenyl optionally substituted as hereinbefore defined (i.e. by B¹) in which one double bond of the alkenyl group is directly attached to the requisite ring of formula I or R₁ represents alkyl substituted with a —OH group α to the point of attachment of the said alkyl group to the requisite ring of formula I and which alkyl group is optionally further substituted as hereinbefore defined (i.e. by B¹) and, in both cases, W represents —NR₇C(O)—, —NR₇S(O)₂—, —NR₇C(O)NR₇—, —NR₇C(O)O— or —NR₇—, —CR₇R₇— or a bond, reaction of a corresponding compound of formula I in which n represents 0 and R₁, represents H with a compound of formula IX,

R_{1b}═O IX

wherein R_{1b} represents alkyl optionally substituted by B¹ as hereinbefore defined, under standard reactions conditions known to those skilled in the art. For example for the preparation of compounds in which R₁ represents alkenyl as defined above, under standard dehydration conditions, e.g. in the presence of a suitable base (such as NaOAc or an appropriate base described hereinafter in respect of process step (vii)) in the presence of a suitable solvent (e.g. glacial acetic acid), e.g. as described in A. Mustafa, W. Musker, A. F. A. M. Shalaby, A. H. Harhash, R. Daguer, Tetrahedron 1964, 20, 25-31. For the preparation of compounds in which R₁ represents alkyl substituted by —OH as defined above, reaction in the presence of a suitable base (e.g. lithium diisopropylamide or another suitable base described in process step (vii) below) in the presence of an appropriate solvent (e.g. anhydrous THF) at room temperature or below (e.g. about 0° C.) under an inert atmosphere. The skilled person will appreciate that for preparation of compounds in which R₁ represents optionally substituted alkenyl as described above, this may involve an intermediate which is the above-mentioned compound of formula I in which R₁ represents alkyl substituted by —OH as defined above (which intermediate may be isolable), which intermediate may need to be transformed to the alkenyl group separately, for example by converting the —OH group to a better leaving group, for example by reaction with trifluoroacetic anhydride or the like optinoall in the presence of a suitable base (e.g. triethylamine) and a catalyst (e.g. DMAP) in an appropriate solvent (e.g. dichloromethane) at below room temperature (such as at about 0° C.) e.g. employing conditions described in Zbirovsky and Seifert, Coll. Czech. Chem. Commun. 1977, 42, 2672-2679;
(v) for compounds of formula I in which n represents 0 and R₁ represents saturated alkyl optionally substituted (i.e. by B¹) as hereinbefore defined, Y represents —S(O)₂ or, preferably, —C(O)— or ═C(R₁₀)— as hereinbefore defined, reduction of a corresponding compound of formula I in which R₁ represents optionally substituted unsaturated alkyl, under standard reaction conditions, for example in the presence of a suitable (e.g. chemoselective) reducing agent such as LiBH₄ or NaBH₄ optionally in the presence of a suitable solvent such as a THF or pyridine (or a mixture thereof, e.g. as described in R. G. Giles, N. J. Lewis, J. K. Quick, M. J. Sasse, M. W. J. Urquhart, L. Youssef, Tetrahedron 2000; 56, 4531-4537. The skilled person will appreciate that the choice of the reducing agent is important in order to achieve the desired reduction selectively (i.e. whilst not reducing other functional groups, such as carbonyl groups, in the compound of formula I). Alternative methods include reduction by hydrogenation under standard conditions, for example in the presence of hydrogen gas or nascent hydrogen, an appropriate solvent (e.g. an alcoholic solvent) and catalyst (e.g. Pd/C);
(vi) for compounds of formula I in which R₆ is alkyl, cycloalkyl or benzyl, all of which are optionally substituted as hereinbefore defined, reaction of a corresponding compound of formula I in which R₆ represents H, with a compound of formula X,

R₆ₐL⁴ X

wherein R₆ₐ represents alkyl, cycloalkyl or benzyl (e.g. which are optionally substituted by one or more groups selected from B¹³, B¹⁴ or B¹⁶, respectively) and L⁴ represents a suitable leaving group such as halo (e.g. iodo or bromo) or a sulfonate group, under standard reaction conditions, for example at around room temperature, in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, 1,8-diazabicyclo[5.4.0]undec-7-ene, sodium hydroxide, or mixtures thereof), an appropriate solvent (e.g. pyridine, dichloromethane, chloroform, tetrahydrofuran, dimethylformamide, triethylamine, dimethylsulfoxide, water or mixtures thereof) and, in the case of biphasic reaction conditions, optionally in the presence of a phase transfer catalyst;
(vii) for compounds of formula I that are substituted with at least one of B¹ to B¹⁸ that represents a —C(O)NR₁₆ₐR_{16b} group, reaction of a corresponding compound of formula I in which that/those (as appropriate) B¹ to B¹⁸ substituents represent —C(O)OR₁₆, with a compound of formula XI,

HNR₁₆ₐR_{16b} XI

or a protected derivative (e.g. a salt) thereof, wherein R₁₆ₐ and R_{16b} are as hereinbefore defined, for example under standard coupling reaction conditions. For example, in the case where R₁₆ represents H, in the presence of a suitable coupling reagent (e.g. 1,1′-carbonyldiimidazole, N,N′-dicyclohexylcarbodiimide, 1-(3-dimethylamino-propyl)-3-ethylcarbodiimide (or hydrochloride thereof), N,N′-disuccinimidyl carbonate, benzotriazol-1-yloxytris(dimethylamino)-phosphonium hexafluorophosphate, 2-(1H-benzotriazol-1-yl)-1,1,3,3-tetramethyluronium hexafluorophosphate, benzotriazol-1-yloxytris-pyrrolidinophosphonium hexafluorophosphate, bromo-tris-pyrrolidinophosphonium hexafluoro-phosphate, 2-(1H-benzotriazol-1-yl)-1,1,3,3-tetramethyluronium tetra-fluorocarbonate) or 1-cyclohexylcarbodiimide-3-propyloxymethyl polystyrene, a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, 1,8-diazabicyclo[5.4.0]undec-7-ene, sodium hydroxide, N-ethyl diisopropylamine, N-(methylpolystyrene)-4-(methylamino)pyridine, potassium bis(trimethylsilyl)-amide, sodium bis(trimethylsilyl)amide, potassium tert-butoxide, lithium diisopropylamide, lithium 2,2,6,6-tetramethylpiperidine or mixtures thereof) and an appropriate solvent (e.g. tetrahydrofuran, pyridine, toluene, dichloromethane, chloroform, acetonitrile or dimethylformamide). Alternatively, for example in the case where R₁₆ is other than H (i.e. —C(O)OR₁₆ represents an ester group), the reaction may be performed in the presence of an appropriate reagent (e.g. trimethylaluminium) in the presence of a suitable solvent (e.g. benzene), for example at elevated temperature (e.g. about 60° C.), e.g. as described in Hwang, K.-J.; O'Neil, J.-P.; Katzenellenbogen, J. A. J. Org. Chem. 1992, 57, 1262;
(viii) for compounds of formula I in which W represents —NR₇C(O)—, —NR₇S(O)₂—, —NR₇C(O)NR₇— or —NR₇C(O)O—, reaction of a corresponding compound of formula I in which W represents —NR₇ and R₅ represents H, with a compound of formula XII,

L⁵W^{x}R₅ XII

wherein W^{x} represents —C(O)—, —S(O)₂, —C(O)NR₇— or —C(O)O—, L⁵ represents a suitable leaving group such as halo (e.g. chloro) and R₅ is as hereinbefore defined, under reaction conditions known to those skilled in the art, for example in the presence of a suitable base (e.g. NaH, NaOH, triethylamine, pyridine, another suitable base mentioned at process step (vii) above or mixtures thereof) and solvent (e.g. pyridine (which may serve as the base and solvent) DMF or dichloromethane (e.g. further in the presence of water and, optionally, a phase transfer catalyst)) for example at room temperature e.g. as described in Hurst, D. T.; Stacey, A. D., Nethercleft, M., Rahim, A., Hamden, M. R. Aust. J. Chem. 1998, 41, 1221; or
(ix) for compounds of formula in which W represents —NR₇C(O)NH—, reaction of a corresponding compound of formula I in which W represents —NR₇ and R₅ represents H, with a compound of formula XIII,

R₅—N═C═O XIII

wherein R₅ is as hereinbefore defined, under standard conditions, for example, in the presence of a suitable solvent (e.g. a polar aprotic solvent such as toluene) and at elevated temperature (e.g. reflux), for example as described in the journal article mentioned in respect of process (viii) above.

Compounds of formula II may be prepared by reaction of a compound of formula XIV,

R₁—X—C(O)H XIV

wherein R₁ and X are as hereinbefore defined, with trichloroacetic acid under standard conditions known to those skilled in the art, for example such as those described in the journal article mentioned in respect of process step (i) (part (A)) above.

Compounds of formula II may be commercially available, prepared under standard conditions or those compounds in which X represents —CH₂—, R₁ represents aryl or heteroaryl optionally substituted as hereinbefore defined and L¹ represents a halo group, reaction of a compound of formula XV,

R_{1c}NH₂ XV

wherein R_{1c} represents aryl or heteroaryl (e.g. optionally substituted by B⁵ and B⁶) to form the corresponding diazonium salt (for example by reaction with sodium nitrite at low temperatures such as at about 0° C.) followed by a compound of formula XVI,

R^{a}—OC(O)CH═CH₂ XVI

wherein R^{a} is as defined above, in the presence of a suitable solvent (e.g. acetone) and a hydrohalic acid which is preferably concentrated (e.g. in the case where L¹ represents chloro, concentrated hydrochloric acid) optionally in the presence of an agent that aids the Michael addition of the halide onto the acrylate/enone such as cuprous oxide.

Compounds of formula III in which L¹ represents a sulfonate group (e.g. a toslyate or mesylate) may be prepared by reaction of a compound corresponding to a compound of formula III but in which L¹ represents —OH with an appropriate sulfonyl chloride (e.g. tosyl chloride or mesyl chloride) under standard conditions known to those skilled in the art, such as those described in respect of preparation of compounds of formula I above (process step (vi) above).

Compounds of formula VI may be prepared by reaction of a compound of formula XVII,

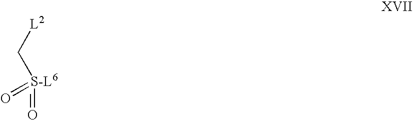

wherein L⁶ represents a suitable leaving group such as halo (e.g. chloro) and L² is as hereinbefore defined, with ammonia (e.g. in gaseous or other form) for example under standard conditions known to those skilled in the art, such as those described in respect of preparation of compounds of formula I above (process step (vi) above) or, preferably, in the presence of diethyl ether at low temperature (e.g. about 0° C.) in which case the skilled person will appreciate that the ammonia additionally serves as a base.

Compounds of formulae IV, V, VII, VIII, IX, X, XI, XII, XIII, XIV, XV, XVI and XVII (and also certain compounds of formula I, II, III and VI) are either commercially available, are known in the literature, or may be obtained either by analogy with the processes described herein (or processes described in references contained herein), or by conventional synthetic procedures, in accordance with standard techniques, from available starting materials using appropriate reagents and reaction conditions.

Substituents, such as R₁, R₅, R₆, X, W and Y in final compounds of formula I or relevant intermediates may be modified one or more times, after or during the processes described above by way of methods that are well known to those skilled in the art. Examples of such methods include substitutions, reductions, oxidations, alkylations, acylations, hydrolyses, esterifications, and etherifications. The precursor groups can be changed to a different such group, or to the groups defined in formula I, at any time during the reaction sequence.

Compounds of formula I may be isolated from their reaction mixtures using conventional techniques.

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3^{rd} edition, T. W. Greene & P. G. M. Wutz, Wiley-Interscience (1999).

As used herein, the term “functional groups” means, in the case of unprotected functional groups, hydroxy-, thiolo-, aminofunction, carboxylic acid and, in the case of protected functional groups, lower alkoxy, N—, O—, S— acetyl, carboxylic acid ester.

The term “cancer” will be understood by those skilled in the art to include one or more diseases in the class of disorders that is characterized by uncontrolled division of cells and the ability of these cells to invade other tissues, either by direct growth into adjacent tissue through invasion, proliferation or by implantation into distant sites by metastasis.

In a preferred embodiment, compounds of formula I are capable of inhibiting the proliferation of cancer cells. By “proliferation” we include an increase in the number and/or size of cancer cells.

Alternatively, or preferably in addition, compounds of formula I are capable of inhibiting metastasis of cancer cells.

By “metastasis” we mean the movement or migration (e.g. invasiveness) of cancer cells from a primary tumour site in the body of a subject to one or more other areas within the subject's body (where the cells can then form secondary tumours). Thus, in one embodiment the invention provides compounds and methods for inhibiting, in whole or in part, the formation of secondary tumours in a subject with cancer. It will be appreciated by skilled persons that the effect of a compound of formula I as described herein on “metastasis” is distinct from any 0.15 effect such compounds may or may not have on cancer cell proliferation.

Advantageously, compounds of formula I may be capable of inhibiting the proliferation and/or metastasis of cancer cells selectively.

By “selectively” we mean that the compound inhibits the proliferation and/or metastasis of cancer cells to a greater extent than it modulates the function (e.g. proliferation) of non-cancer cells. Preferably, the compound inhibits the proliferation and/or metastasis of cancer cells only.

The medicaments are suitable for use in the treatment of any cancer type. For example, the cancer cells may be selected from the group consisting of cancer cells of the breast, bile duct, brain, colon, stomach, reproductive organs, thyroid, hematopoetic system, lung and airways, skin, gallbladder, liver, nasopharynx, nerve cells, kidney, prostate, lymph glands and gastrointestinal tract. Preferably, the cancer is selected from the group of colon cancer (including colorectal adenomas), breast cancer (e.g. postmenopausal breast cancer), endometrial cancer, cancers of the hematopoetic system (e.g. leukemia, lymphoma, etc), thyroid cancer, kidney cancer, oesophageal adenocarcinoma, ovarian cancer, prostate cancer, pancreatic cancer, gallbladder cancer, liver cancer and cervical cancer. More preferably, the cancer is selected from the group of colon, breast and prostate cancer.

Preferably, the cancer cells are breast cancer cells.

According to a further aspect of the invention there is provided a method of treatment of cancer, which method comprises the administration of an effective amount of a compound of formula I to a patient in need of such treatment.

For the avoidance of doubt, in the context of the present invention, the terms “treatment”, “therapy” and “therapy method” include the therapeutic, or palliative, treatment of patients in need of as well as the prophylactic treatment and/or diagnosis of patients which are susceptible to, cancer.

“Patients” include mammalian (including human) patients.

The term “effective amount” refers to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

Novel compounds of formula I as hereinbefore defined are useful as medicaments and are therefore indicated as pharmaceuticals.

In accordance with the invention, compounds of formula I may be administered alone, but are preferably administered orally, intravenously, intramuscularly, cutaneously, subcutaneously, transmucosally (e.g. sublingually or buccally), rectally, transdermally, nasally, pulmonarily (e.g. tracheally or bronchially), topically, by any other parenteral route, in the form of a pharmaceutical preparation comprising the compound in a pharmaceutically acceptable dosage form. Preferred modes of delivery include oral, intravenous, cutaneous or subcutaneous, nasal, intramuscular, or intraperitoneal delivery.

Compounds of formula I will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations, may be found in, for example, Remington The Science and Practice of Pharmacy, 19th ed., Mack Printing Company, Easton, Pa. (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science 249, 1527 (1990).

Otherwise, the preparation of suitable formulations may be achieved non-inventively by the skilled person using routine techniques and/or in accordance with standard and/or accepted pharmaceutical practice.

Another aspect of the present invention includes a pharmaceutical composition comprising a therapeutically effective amount of a novel compound of formula I as hereinbefore defined in combination with a pharmaceutically acceptable excipient, such as an adjuvant, diluent or carrier.

The amount of compound of formula I in the formulation will depend on the severity of the condition, and on the patient, to be treated, as well as the compound(s) which is/are employed, but may be determined non-inventively by the skilled person.

Depending on the disorder, and the patient, to be treated, as well as the route of administration, compounds of formula I may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a compound of formula I (or, if employed, a corresponding amount of a pharmaceutically acceptable salt or prodrug thereof).

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

The compounds of formula I may be used or administered in combination with one or more additional drugs useful in the treatment of cancer, in combination therapy.

According to a further aspect of the invention, there is provided a combination product comprising:

(A) a compound of formula I; and
(B) another therapeutic agent useful in the treatment of cancer, wherein each of components (A) and (B) is formulated in admixture with a pharmaceutically-acceptable adjuvant, diluent or carrier.

Such combination products provide for the administration of compound of formula I in conjunction with the other therapeutic agent, and may thus be presented either as separate formulations, wherein at least one of those formulations comprises compound of formula I, and at least one comprises the other therapeutic agent, or may be presented (i.e. formulated) as a combined preparation (i.e. presented as a single formulation including compound of formula I and the other therapeutic agent).

Thus, there is further provided:

(1) a pharmaceutical formulation including a compound of formula I; another therapeutic agent useful in the treatment of cancer; and a pharmaceutically-acceptable adjuvant, diluent or carrier; and
(2) a kit of parts comprising components:
(a) a pharmaceutical formulation including a compound of formula I in admixture with a pharmaceutically-acceptable adjuvant, diluent or carrier; and
(b) a pharmaceutical formulation including another therapeutic agent useful in the treatment of cancer in admixture with a pharmaceutically-acceptable adjuvant, diluent or carrier,
   which components (a) and (b) are each provided in a form that is suitable for administration in conjunction with the other.

Components (a) and (b) of the kit of parts described herein may be administered simultaneously or sequentially.

The method/use described herein may have the advantage that, in the treatment of cancer, it may be more convenient for the physician and/or patient than, be more efficacious than, be less toxic than, have a broader range of activity than, be more potent than, produce fewer side effects than, or that it may have other useful pharmacological properties over, similar methods (treatments) known in the prior art for use in the treatment of cancer or otherwise.

The invention is illustrated by the following examples in which error bars denote SEM and the following abbreviations are employed:

LA—linolenic acid
DMSO—dimethyl sulfoxide.

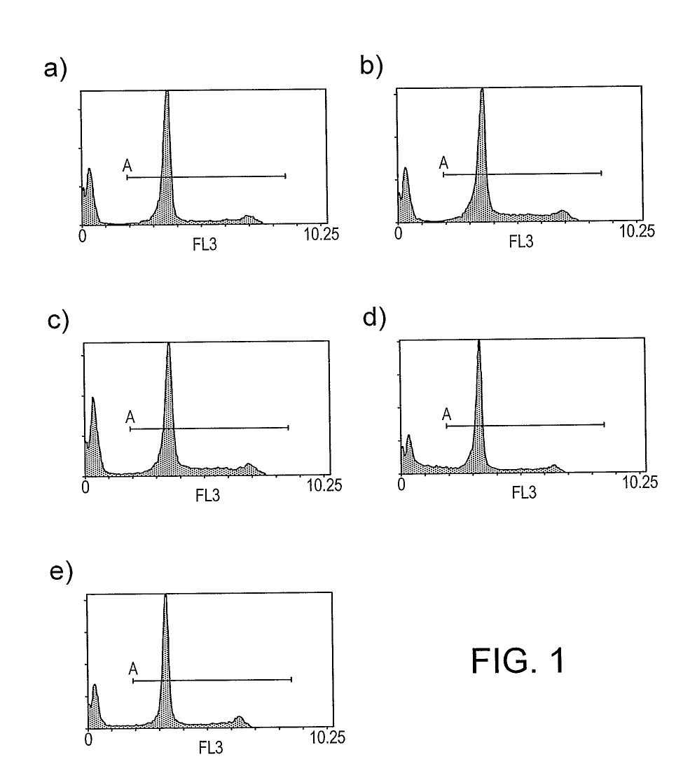

FIGS. 1a to 1e are representative examples of cell cycle analysis using Flow Cytometer. Cells were incubated with or without linolenic acid and the compound of Example 95 below (Compound X) for 24 hours. Histograms represent accumulated events and their distribution in the cell cycle by intensity of PI staining (FL3). (a) untreated, (b) LA 100 μM, (c) LA 100 μM+Compound X 10 μM, (d) Compound X 10 μM, (e) DMSO 0.2%.

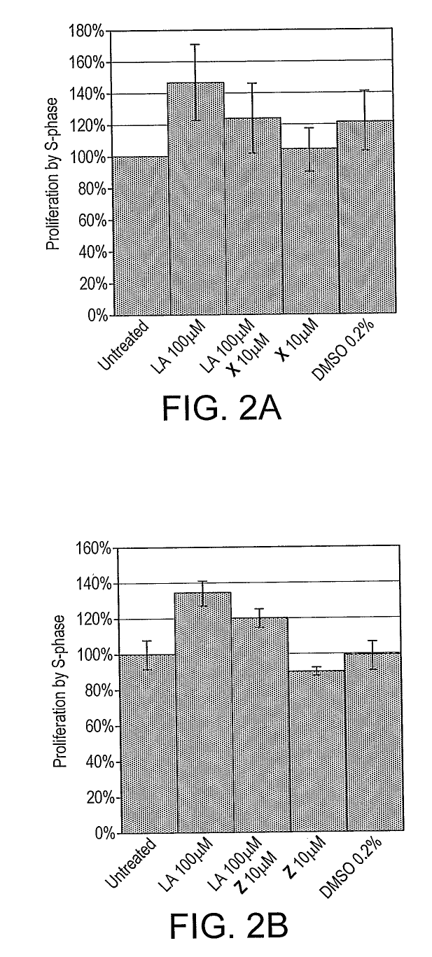

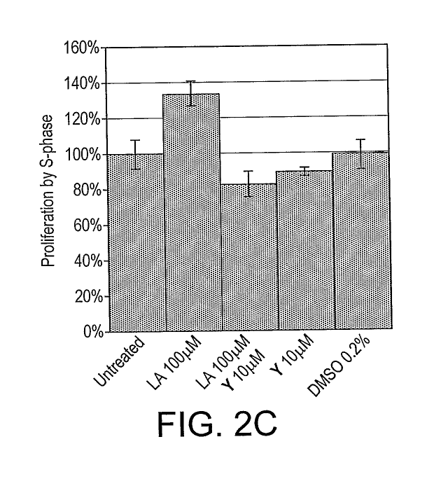

FIG. 2A is a histogram summarizing 4 experiments where one compound is identified and verified as an FFA antagonist. Cells were incubated with or without linolenic acid and the Compound X for 24 hours at indicated concentrations. Cells in S-phase from untreated sample were set to 100% in each experiment.

FIGS. 2B and 2C are histograms where compounds are identified and verified as FFA antagonists. Cells were incubated with or without linolenic acid and the compound of Examples 4 and 6 below (Compound Z and Compound Y, respectively) for 24 hours at indicated concentrations. Cells in S-phase from untreated sample were set to 100% in each experiment (n=2).

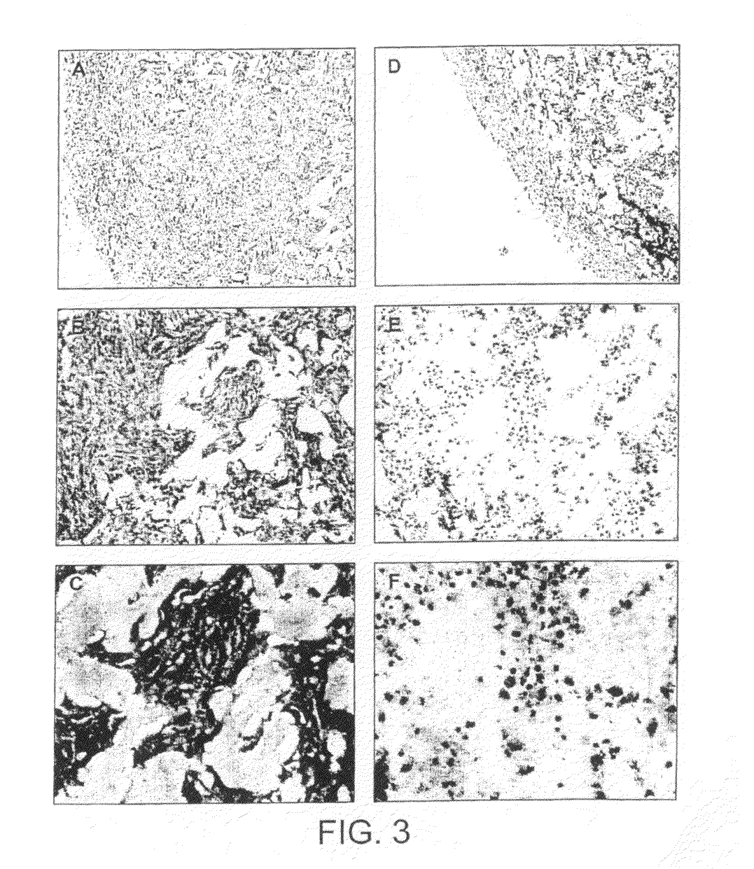

FIGS. 3A to 3F show hematoxylin stained sections from tumors dissected from vehicle or test compound treated mice.

### EXAMPLES

Where no preparative routes are includes, the relevant example is commercially available (e.g. from Chemical Diversity, San Diego, Calif., USA or other available commercial sources).

### Example 1

5-Benzyl-2-(phenylimino)thiazolidin-4-one

### Example 2

5-(4-Methylbenzyl)-2-(4-chlorophenylimino)thiazolidin-4-one

### Example 3

5-(4-Chlorobenzyl)-2-(4-chlorophenylimino)thiazolidin-4-one

### Example 4

### 5-(3-(Trifluoromethyl)benzyl)-2-(p-tolylimino)thiazolidin-4-one

### (a) Methyl 2-chloro-3-(3-(trifluoromethyl)phenyl)propanoate

A solution of sodium nitrite (0.47 g, 6.82 mmol) in water (1.4 mL) was added dropwise to a solution of 3-trifluoromethylaniline (0.77 mL, 6.21 mmol) in concentrated hydrochloric acid and acetone (14 mL), which mixture was prior cooled under an ice-water bath. The mixture was stirred at 0° C. for 10 min. After addition of methyl acrylate (3.37 mL, 37.4 mmol), cuprous oxide (40 mg) was added portionwise to the mixture at 40° C. The mixture was stirred at 35° C. for 20 min and then washed twice with equal amounts of water and ethyl acetate (50 mL). The organic layer was dried with MgSO₄, filtered and concentrated. The crude oil was purified by silica gel chromatography using chloroform as eluent to give the sub-title compound (1.22 g, 4.58 mmol, 74%) as yellow oil. ES-MS m/z 289.1 (MNa+). ¹H NMR δ(CDCl₃): 3.24 (dd, 1H), 3.43 (dd, 1H), 3.76 (s, 3H), 4.46 (dd, 1H), 7.4-7.6 (m, 4H).

### (b) 5-(3-(Trifluoromethyl)benzyl)-2-(p-tolylimino)thiazolidin-4-one

A mixture of methyl 2-chloro-3-(3-(trifluoromethyl)phenyl)propanoate (0.61 g, 2.29 mmol; see step (a) above), N-(p-methylphenyl) thiourea (698 mg, 4.2 mmol) and sodium acetate (212 mg, 2.54 mmol) in ethanol (5.0 mL) was refluxed for 8 hours and then concentrated. The crude product was purified by silica gel chromatography using toluene:ethyl acetate (3:2) as eluent followed by re-crystallisation from hot methanol to give the title compound (170 mg, 0.47 mmol, 21%) as a white solid. LC-MS (A) t_{R}: 6.26 min, m/z 365.2 (MH+). ¹H NMR: δ(DMSO-d₆): 2.27 (s, 3H), 3.14 (m, 1H), 3.46 (dd, 1H), 4.75 (m, 1H), 6.80 (m, 1H), 7.12 (m, 2H), 7.56 (m, 5H).

### Example 5

### 5-(3-(Trifluoromethyl)benzyl)-2-(4-isopropylphenylimino)thiazolidin-4-one

The title compound was prepared in accordance with Example 4. The title compound was purified by flash chromatography and recrystallised from hot methanol to give 167 mg of the title compound as a white solid. LC-MS (A) t_{R}: 7.03 min, m/z 393.4 (MH+). ¹H NMR: δ(DMSO-d₆): 1.15 (d, 6H), 2.83 (m, 1H), 3.15 (m, 1H), 3.45 (ddd, 1H), 4.75 (m, 1H), 6.83 (d, 1H), 7.30 (dd, 2H), 7.45-7.65 (m, 5H).

### Example 6

### 5-(3-(Trifluoromethyl)benzyl)-2-(4-chlorophenylimino)thiazolidin-4-one

The title compound was prepared in accordance with Example 4. The title compound was purified by flash chromatography and recrystallised from hot methanol to give 271 mg of the title compound as a white solid. LC-MS (A) t_{R}: 6.9 min, m/z 385.4 (MH+). ¹H NMR: δ(DMSO-d₆): 3.2 (m, 1H), 3.6 (big HDO signal), 4.8 (m, 1H), 6.85 (d, 1H), 7.4 (dd, 2H), 7.5-7.7 (m, 6H).

### Example 7

### 5-(3-(Trifluoromethyl)benzyl)-2-(4-methoxyphenylimino)thiazolidin-4-one

The title compound was prepared in accordance with Example 4. The title compound was purified by flash chromatography and recrystallised from hot methanol to give 137 mg of the title compound as a white solid. LC-MS (A) t_{R}: 6.25 min, m/z 381.2 (MH+). ¹H NMR: δ(DMSO-d₆): 3.12 (dd, 1H), 3.45 (ddd, 1H), 4.74 (dd, 1H), 6.86-6.95 (m, 3H), 7.50-7.63 (m, 5H).

### Example 8

### 5-(3-(Trifluoromethyl)benzyl)-2-(phenylimino)thiazolidin-4-one

The title compound was prepared in accordance with Example 4. The title compound was purified by flash chromatography and recrystallised from hot methanol to give 289 mg of the title compound as a white solid. LC-MS (A) t_{R}: 6.42 min, m/z 351.4 (MH+). ¹H NMR: δ(DMSO-d₆): 3.1-3.5 (m, 2H), 4.76 (dd, 1H), 6.86 (d, 1H), 7.11 (m, 1H), 7.23 (m, 2H), 7.57 (m, 5H).

### Example 9

### 5-(4-Fluorobenzyl)-2-(phenylimino)thiazolidin-4-one

The title compound was prepared in accordance with Example 4. The title compound was purified by flash chromatography and recrystallised from hot methanol to give 181 mg of the title compound as a white solid. LC-MS (B) t_{R}: 1.57 min, m/z 301.3 (MH+). ¹H NMR: δ(DMSO-d₆): 3.00 (dd, 1H), 3.15-3.40 (m, 2H), 4.69 (dd, 1H), 6.90 (m, 1H), 7.11 (m, 3H), 7.30 (m, 4H), 7.62 (d, 1H).

### Example 10

### 5-(4-Fluorobenzyl)-2-(p-tolylimino)thiazolidin-4-one

The title compound was prepared in accordance with Example 4. The title compound was purified by flash chromatography and recrystallised from hot methanol to give 144 mg of the title compound as a white solid. LC-MS (B) t_{R}: 1.62 min, m/z 315.2 (MH+). ¹H NMR: δ(DMSO-d₆): 2.23 (s, 3H), 2.99 (m, 1H), 3.12-3.41 (m, 2H), 4.65 (m, 1H), 6.80 (m, 1H), 7.11 (m, 4H), 7.25 (m, 2H), 7.49 (d, 1H).

### Example 11

### 2-(4-Chlorophenylimino)-5-(4-fluorobenzyl)thiazolidin-4-one

The title compound was prepared in accordance with Example 4. The title compound was purified by flash chromatography and recrystallised from hot methanol to give 175 mg of the title compound as a white solid. LC-MS (B) t_{R}: 1.75 min, m/z 335.2 (MH+). ¹H NMR: δ(DMSO-d₆): 3.0 (dd, 1H), 3.3 (m, 1H, HDO), 4.7 (dd, 1H), 6.9-7.7 (m, 8H).

### Example 12

### 5-(4-Fluorobenzyl)-2-(4-methoxyphenylimino)thiazolidin-4-one

The title compound was prepared in accordance with Example 4. The title compound was purified by flash chromatography and recrystallised from hot methanol to give 166 mg of the title compound as a white solid. LC-MS (B) t_{R}: 1.51 min, m/z 331.1 (MH+). ¹H NMR: δ(DMSO-d₆): 2.99 (dd, 1H), 3.36 (m, 1H, HDO), 3.72 (s, 3H), 4.65 (b, 1H), 6.90 (m, 3H), 7.10 (m, 2H), 7.25 (m, 2H), 7.40 (d, 1H).

### Example 13

### 5-(4-Fluorobenzyl)-2-(4-isopropylphenylimino)thiazolidin-4-one

The title compound was prepared in accordance with Example 4. The title compound was purified by flash chromatography and recrystallised from hot methanol to give 55 mg of the title compound as a white solid. LC-MS (A) t_{R}: 7.30 min, m/z 343.2 (MH+). ¹H NMR: δ(DMSO-d₆): 1.18 (d, 6H), 2.82 (m, 1H), 3.10 (m, 1H), 3.15-3.41 (m, 1H), 4.66 (dd, 1H), 6.83 (m, 1H), 7.1-7.3 (m, 6H), 7.51 (d, 1H).

### Example 14

### 5-(4-(Trifluoromethyl)benzyl)-2-(p-tolylimino)thiazolidin-4-one

The title compound was prepared in accordance with Example 4. The title compound was purified by flash chromatography and recrystallised from hot methanol to give 242 mg of the title compound as a white solid. LC-MS (A) t_{R}: 7.50 min, m/z 365.2 (MH+). ¹H NMR: δ(DMSO-d₆): 2.25 (s, 3H), 3.10 (m, 1H), 3.36 (m, 1H), 4.72 (m, 1H), 6.80 (m, 1H), 7.12 (dd, 2H), 7.46 (m, 3H), 7.63 (m, 2H).

### Example 15

### 5-(4-Methoxybenzyl)-2-(p-tolylimino)thiazolidin-4-one

The title compound was prepared in accordance with Example 4. The title compound was purified by flash chromatography and recrystallised from hot methanol to give 282 mg of the title compound as a white solid. LC-MS (A) t_{R}: 6.45 min, m/z 327.4 (MH+). ¹H NMR: δ(DMSO-d₆): 2.25 (s, 3H), 2.90 (dd, 1H), 3.33 (m, 1H), 3.70 (s, 3H), 4.60 (dd, 1H), 6.83 (m, 3H), 7.12 (m, 4H), 7.50 (d, 1H).

### Example 16

### 5-Benzyl-2-(phenylimino)thiazolidin-4-one

The title compound was prepared in accordance with Examples 26 and 65 below. The title compound was purified by flash chromatography yielding 27 mg of the title compound. LC-MS (A) t_{R}: 8.50 min. ES-MS m/z: 283.2 (MH+). ¹H NMR: δ(DMSO-d₆): 3.00 (dd, 1H), 3.40 (m, 1H), 4.75 (dd, 1H), 6.90 (d, 1H), 7.05-7.45 (m, 8H), 7.65 (d, 1H).

### Example 17

### 5-(3-(Trifluoromethyl)benzyl)-2-(4-fluorophenylimino)thiazolidin-4-one

The title compound was prepared in accordance with Example 4. The title compound was purified by flash chromatography and recrystallised from hot methanol to give 78 mg of the title compound as a white powder. LC-MS (A) t_{R}: 9.14 min. ES-MS m/z: 369.0 (MH+). ¹H NMR: δ(DMSO-d₆): 3.10-3.25 (m, 1H), 3.45 (ddd, 1H), 4.80 (m, 1H), 6.9 (m, 1H), 7.10-7.30 (m, 2H), 7.50-7.75 (m, 5H).

### Example 18

### 5-(3-(Trifluoromethyl)benzyl)-2-(4-bromophenylimino)thiazolidin-4-one

The title compound was prepared in accordance with Example 4. The title compound was purified by flash chromatography and recrystallised from hot methanol to give 803 mg of the title compound as an off-white powder. LC-MS (A) t_{R}: 9.38 min. ES-MS m/z: 431.2 (MH+). ¹H NMR: δ(DMSO-d₆): 3.20 (m, 1H), 3.40 (dd, 1H), 4.75 (m, 1H), 7.40-7.60 (m, 7H).

### Example 19

### 2-(3,4-Dichlorophenylimino-5-(3-(trifluoromethyl)benzyl)thiazolidin-4-one

The title compound was prepared in accordance with Example 4. The title compound was purified by flash chromatography and recrystallised from hot methanol to give 67 mg of the title compound as a white powder. LC-MS (A) t_{R}: 9.14 min. ES-MS m/z: 369.0 (MH+). ¹H NMR: δ(DMSO-d₆): 3.15 (app. t, 1H), 3.45 (m, 1H), 4.80 (m, 1H), 6.85 (d, 1H), 7.10 (s, 1H), 7.50-7.70 (5H), 8.10 (m, 1H).

### Example 20

### 2-(2,4-Dichlorophenylimino)-5-(3-(trifluoromethyl)benzyl)thiazolidin-4-one

The title compound was prepared in accordance with Example 4. The title compound was purified by flash chromatography and recrystallised from hot methanol to give 68 mg of the title compound as an off-white powder. LC-MS (A) t_{R}: 9.52 min. ES-MS m/z: 419.0 (MH+). ¹H NMR: δ(DMSO-d₆): 3.20 (m, 1H), 3.40 (dd, 1H), 4.80 (dd, 1H), 6.95 (d, 1H), 7.35 (d, 1H), 7.50-7.65 (m, 4H).

### Example 21

### 4-(5-(3-(Trifluoromethyl)benzyl)-4-oxothiazolidin-2-ylideneamino)benzonitrile

The title compound was prepared in accordance with Example 4. The title compound was purified by flash chromatography and recrystallised from hot methanol to give 45 mg of the title compound as a white powder. LC-MS (A) t_{R}: 8.98 min. ES-MS m/z: 376.2 (MH+). ¹H NMR: δ(DMSO-d₆): 3.20 (dd, 1H), 3.50 (bs, 1H), 4.85 (bs, 1H), 7.00 (bs, 1H), 7.50-8.00 (m, 7H).

### Example 22

### Ethyl 4-(5-(3-(trifluoromethyl)benzyl)-4-oxothiazolidin-2-ylideneamino)benzoate

The title compound was prepared in accordance with Example 4. The title compound was purified by flash chromatography and recrystallised from hot ethyl acetate to give 560 mg of the title compound as a white crystals. LC-MS (A) t_{R}: 8.77 min. ES-MS m/z 423.2 (MH+). ¹H NMR: δ (400 MHz) (CDCl₃): 1.50 (t, 3H), 3.31 (dd, 1H), 3.67 (dd, 1H), 4.48 (q, 2H), 4.58 (dd, 1H), 7.17-7.23 (m, 2H), 7.48-7.69 (m, 4H), 8.14 (d, 2H) ppm.

### Example 23

### 4-(5-(3-(Trifluoromethyl)benzyl)-4-oxothiazolidin-2-ylideneamino)benzoic Acid

Ethyl 4-(5-(3-(trifluoromethyl)benzyl)-4-oxothiazolidin-2-ylideneamino)benzoate (100 mg, 0.24 mmol; see Example 22) was dissolved in a dioxane/water mixture (4:1, 5 mL), and 1.0 M aqueous LiOH (0.5 mL) was added. The reaction mixture was refluxed for 6 hours and then acidified with 1.0 M aqueous HCl. The precipitate that had formed was filtered off to give 93 mg (0.24 mmol, 99%) of the title compound as a white solid. LC-MS (A) t_{R}: 8.32 min. ES-MS m/z 395.0 (MH+). ¹H NMR: δ (400 MHz) (DMSO-d₆): 3.26-3.62 (m, 2H), 4.87-4.95 (m, 1H), 6.97-7.08 (m, 2H), 7.61-8.09 (m, 6H) ppm.

### Example 24

### 4-(5-(3-(Trifluoromethyl)benzyl)-4-oxothiazolidin-2-ylideneamino)benzamide

To a solution of NH₄Cl (324 mg, 6.00 mmol) in anhydrous benzene (6 ml) was added a 25% solution (3.0 ml, 6.00 mmol) of trimethylaluminium in hexane at 0° C. After removal of the ice bath, the reaction mixture was stirred for 1.5 hours until no gas evolution was observed. To this aluminium reagent, a solution of ethyl 4-(5-(3-(trifluoromethyl)benzyl)-4-oxothiazolidin-2-ylideneamino)benzoate (393 mg, 1.00 mmol; see Example 23) in benzene (2 ml) was added at room temperature. The yellow solution was stirred at 60° C. for 1.5 hours, cooled to room temperature, and CH₂Cl₂ and water were added. The organic phase was dried over MgSO₄, filtered and concentrated in vacuum. The crude product was purified by silica gel column chromatography using a gradient of petroleum ether/EtOAc (10-50%) as eluent to render 56 mg (0.14 mmol, 14% yield) of the title compound as a white solid. LC-MS (A) t_{R}: 8.32 min. ES-MS m/z 394.2 (MH+). ¹H NMR: δ (400 MHz) (DMSO-d₆): 3.20-3.35 (m, 1H), 3.44-3.66 (m, 1H), 4.87-4.98 (m, 1H), 6.94-7.05 (m, 1H), 7.29-7.43 (m, 1H), 7.58-8.09 (m, 8H) ppm.

### Example 25

### 5-(3-(Trifluoromethyl)benzyl)-2-(m-tolylimino)thiazolidin-4-one

The title compound was prepared in accordance with Example 4. The title compound was purified by flash chromatography and recrystallised from hot methanol to give 220 mg of the title compound as a white powder. LC-MS (A) t_{R}: 9.52 min. ES-MS m/z: 365 (MH+). ¹H NMR: δ(DMSO-d₆): 7.10-7.61 (m, 8H), 3.86 (t, 1H), 3.56 (m, 1H), 3.30 (m, 1H), 2.35 (s, 3H).

### Example 26

### 2-(4-Chlorophenylimino)-5-(4-fluoro-3-(trifluoromethyl)benzyl)thiazolidin-4-one

### (a) 2-(4-Chlorophenylimino)thiazolidin-4-one

A mixture of ethyl 2-bromoacetate (0.25 mL, 2.29 mmol), N-(4-chlorophenyl)thiourea (2.29 mmol) and sodium acetate (212 mg, 2.54 mmol) in ethanol (5 mL) was refluxed overnight. The mixture was concentrated, diluted with dichloromethane and washed with water. The organic layer was dried with MgSO₄, filtered and concentrated. The crude product was purified by silica gel chromatography using toluene:ethyl acetate (2:1) as eluent (441 mg) and recrystallized from methanol to give 178 mg (0.86 mmol, 38%) of the sub-title compound. LC-MS (A) t_{R}: 4.68 min, m/z 207.2 (MH+). ¹H NMR: δ(DMSO-d₆): 2.26 (s, 3H), 3.84 (d, 2H), 6.69 (d, 1H), 7.16 (d, 2H), 7.57 (d, 1H).

### (b) 2-(4-Chlorophenylimino)-5-(4-fluoro-3-(trifluoromethyl)-benzylidene)thiazol-idin-4-one

A mixture of 2-(4-chlorophenylimino)thiazolidin-4-one (0.48 mmol; see step (a) above), benzaldehyde (0.73 mmol) and NaOAc (62 mg, 0.75 mmol) in 2 mL glacial AcOH was refluxed for 21 h. The solvent was evaporated, and the crude product was purified by silica gel column chromatography using toluene:acetone 3:1 as eluent yielding 120 mg (78%) of the sub-title compound as a brown powder. LC-MS (A) t_{R}: 9.30 min. ES-MS m/z: 323 (MH+).

### (c) 2-(4-Chlorophenylimino)-5-(4-fluoro-3-(trifluoromethyl)benzyl)thiazolidin-4-one

A mixture of 2-(4-chlorophenylimino)-5-(4-fluoro-3-(trifluoromethyl)benzyl-idene)thiazolidin-4-one (61.7 mg, 0.154 mmol; see step (b) above) and pyridine (0.5 mL) in THF (0.4 mL) was heated in a closed screw-cap tube at 70° C. for 2 hours. LC-MS monitoring showed no traces of the desired product. Sodium borohydride (40 mg, 1.06 mmol) was added and the mixture was heated overnight. The reaction was quenched with acetic acid (2 mL), diluted with ethyl acetate, washed with water and concentrated in vacuum. The crude product (126.4 mg) was purified by silica gel column chromatography using petroleum ether:ethyl acetate (2:1) as eluent and by subsequent precipitation of impurities using ethyl acetate/petroleum ether twice yielding 30 mg (0.074 mmol, 48% yield) of the title compound as an oil. LC-MS (A) t_{R}: 10.88 min. (B) t_{R}: 0.68 min. m/z 403.3/405.3 (MH+).

### Example 27

### 5-(3-(Trifluoromethyl)benzyl)-2-(p-tolylimino)-3-methylthiazolidin-4-one

A mixture of 5-(3-(trifluoromethyl)benzyl)-2-(p-tolylimino)thiazolidin-4-one (250 mg, 0.686 mmol), sodium carbonate (145 mg, 1.37 mmol) and methyl iodide (127 μL, 1.37 mmol) in DMF (2.5 mL) was stirred at room temperature overnight. The mixture was diluted with dichloromethane and washed with water. The organic layer was dried with MgSO₄, filtered and concentrated. The crude product was purified by silica gel chromatography using toluene:ethyl acetate (2:1) as eluent to yield the title compound (99 mg, 0.262 mmol, 38%). LC-MS (B) t_{R}: 0.98 min (256 nm). ¹H NMR: δ(DMSO-d₆): 2.42 (s, 3H), 3.11 (d, 1H), 3.28 (s, 3H), 3.33 (dd, 2H), 7.20-7.33 (m, 6H), 7.38 (t, 1H), 7.53 (d, 1H).

### Example 28

### 5-(3-(Trifluoromethyl)benzyl)-2-(N-methyl-N-phenylamino)thiazol-4(5H)-one

The title compound was prepared in accordance with Example 4. The title compound was purified by flash chromatography and recrystallised from hot methanol to give 237 mg of the title compound as a white powder. LC-MS (A) t_{R}: 8.82 min. ES-MS m/z: 365 (MH+). ¹H NMR: δ(DMSO-d₆): 7.61-7.10 (m, 6H), 7.30-7.10 (m, 3H), 4.4 (t, 1H), 3.55 (m, 1H), 3.15 (m, 1H), 2.35 (s, 3H).

### Example 29

### 5-(3-(Trifluoromethyl)benzyl)-2-(N-methyl-N-p-tolylamino)thiazol-4(5H)-one

The title compound is prepared in accordance with the procedures described herein.

### Example 30

### 5-(4-Fluorobenzyl)-2-(N-methyl-N-(pyridin-2-yl)amino)thiazol-4(5H)-one

The title compound is prepared in accordance with the procedures described herein.

### Example 31

### 2-(2-(N-Methyl-N-p-tolylamino)-4,5-dihydro-4-oxothiazol-5-yl)-N-p-tolylacetamide

The title compound is prepared in accordance with the procedures described herein.

### Example 32

### 5-(3-(Trifluoromethyl)benzyl-2-(N-benzyl-N-p-tolylamino)thiazol-4(5H)-one

The title compound is prepared in accordance with the procedures described herein.

### Example 33

### 5-(4-Fluorobenzyl)-2-(N-benzyl-N-(pyridin-2-yl)amino)thiazol-4(5H)-one

The title compound is prepared in accordance with the procedures described herein.

### Example 34

### 2-(2-(N-Benzyl-N-p-tolylamino)-4,5-dihydro-4-oxothiazol-5-yl)-N-p-tolylacetamide

The title compound is prepared in accordance with the procedures described herein.

### Example 35

### 5-(3-(Trifluoromethyl)benzyl)-2-(N-phenyl-N-p-tolylamino)thiazol-4(5H)-one

The title compound is prepared in accordance with the procedures described herein.

### Example 36

### 5-(4-Fluorobenzyl)-2-(N-phenyl-N-(pyridin-2-yl)amino)thiazol-4(5H-one

The title compound is prepared in accordance with the procedures described herein.

### Example 37

### 2-(2-(N-phenyl-N-p-tolylamino)-4.5-dihydro-4-oxothiazol-5-yl)-N-p-tolylacetamide

The title compound is prepared in accordance with the procedures described herein.

### Example 38

### 5-(3-(Trifluoromethyl)benzylidene)-2-(phenylimino)thiazolidin-4-one

The title compound was prepared in accordance with Examples 26 and 65, steps (a) and (b). The product precipitated from the reaction mixture, was filtered off washed with AcOH and toluene and was dried in vacuo to yield 50 mg of the title compound as a yellow powder. LC-MS (A) t_{R}: 9.46 min. ES-MS m/z: 349.4 (MH+). ¹H NMR: δ(DMSO-d₆): 7.05 (d, 1H), 7.22 (t, 1H), 7.40 (m, 2H), 7.70-8.00 (m, 5H).

### Example 39

### 5-(3-(Trifluoromethyl)benzylidene)-2-(p-tolylimino)thiazolidin-4-one

The title compound was prepared in accordance with Examples 26 and 65, steps (a) and (b). The product precipitated from the reaction mixture, was filtered off, washed with AcOH and toluene and was dried in vacuo to yield 47 mg of the title compound as a yellow powder. LC-MS (A) t_{R}: 9.32 min. ES-MS m/z: 363.2 (MH+). ¹H NMR: δ(DMSO-d₆): 2.30 (s, 3H), 6.95 (m, 1H), 7.25 (t, 2H), 7.60-7.85 (m, 4H), 7.95 (m, 2H).

### Example 40

### 5-(4-Fluorobenzylidene)-2-(phenylimino)thiazolidin-4-one

The title compound was prepared in accordance with Examples 26 and 65, steps (a) and (b). The product precipitated from the reaction mixture, was filtered off, washed with AcOH and toluene and was dried in vacuo to yield 39 mg of the title compound as a yellow powder. LC-MS (A) t_{R}: 9.14 min. ES-MS m/z: 299.0 (MH+). ¹H NMR: δ(DMSO-d₆): 7.05 (d, 1H), 7.20 (t, 1H), 7.30-7.50 (m, 4H), 7.55-7.80 (m, 3H).

### Example 41

### 5-(4-Fluorobenzylidene)-2-(p-tolylimino)thiazolidin-4-one

The title compound was prepared in accordance with Examples 26 and 65, steps (a) and (b). The product precipitated from the reaction mixture, was filtered off, washed with AcOH and toluene and was dried in vacuo to yield 49 mg of the title compound as a yellow powder. ¹H NMR: δ(DMSO-d₆): 2.35 (s, 3H), 7.00 (app. s, 1H), 7.25 (t, 2H), 7.35 (t, 1H), 7.45 (t, 1H), 7.60 (t, 1H), 7.65 (t, 1H), 7.65-7.75 (m, 3H).

### Example 42

### 5-Benzylidene-2-(phenylimino)thiazolidin-4-one

The title compound was prepared in accordance with Examples 26 and 65, steps (a) and (b). The product precipitated from the reaction mixture, was filtered off, recrystallised from acetic acid (2×), washed with toluene and dried in vacuo to give 442 mg of the title compound. ¹H NMR: δ(CD₃CN-d₃): 7.03 (d, 2H), 7.19 (t, 2H), 7.44 (m, 2H), 7.63 (m, 2H), 7.71 (s, 1H), 7.78 (d, 2H).

### Example 43

### 2-(p-Tolylimino)-5-benzylidenethiazolidin-4-one

The title compound was prepared in accordance with Examples 26 and 65, steps (a) and (b). The product precipitated from the reaction mixture, was filtered off, washed with AcOH and toluene and was dried in vacuo to yield 43 mg of the title compound as a yellow powder. ¹H NMR: δ(DMSO-d₆): 2.40 (s, 3H), 7.95 (d, 1H), 7.25 (t, 2H), 7.37-7.75 (6H).

### Example 44

### 5-(3-(Trifluoromethyl)benzylidene)-2-(4-chlorophenylimino)thiazolidin-4-one

The title compound was prepared in accordance with Examples 26 and 65, steps (a) and (b).

### Example 45

### 2-(4-Chlorophenylimino)-5-benzylidenethiazolidin-4-one

The title compound was prepared in accordance with Examples 26 and 65, steps (a) and (b). The product precipitated from the reaction mixture, was filtered off, washed with AcOH and toluene and was dried in vacuo to yield 83 mg of the title compound as a yellow powder. LC-MS (A) t_{R}: 9.46 min. ES-MS m/z: 314.8 (MH+). ¹H NMR: δ(DMSO-d₆): 7.05 (d, 2H), 7.40-7.60 (m, 4H), 7.65 (m, 2H), 7.70 (s, 1H), 8.80 (d, 1H).

### Example 46

### 2-(4-Chlorophenylimino)-5-(4-fluoro-3-(trifluoromethyl)benzylidene)thiazolidin-4-one

The title compound was prepared in accordance with Examples 26 and 65, steps (a) and (b). The product precipitated from the reaction mixture, was filtered off and recrystallised from acetic acid to give 83 mg of the title compound. LC-MS (A) t_{R}: 11.03 min. (B) t_{R}: 0.82 min. m/z 401.3/403.2 (MH+).

### Example 47

### 2-(4-Methylbenzyl)-5-(3-trifluoromethyl-benzyl)-thiazol-4-one

The title compound is prepared in accordance with the procedures described herein.

### Example 48

### 5-(4-Fluorobenzyl)-2-pyridin-2-ylmethylthiazol-4-one

The title compound is prepared in accordance with the procedures described herein.

### Example 49

### 2-[2-(4-Methylbenzyl)-4-oxo-4,5-dihydrothiazol-5-yl]-N-p-tolyl-acetamide

The title compound is prepared in accordance with the procedures described herein.

### Example 50

### 2-(1-p-Tolylethyl)-5-(3-trifluoromethylbenzyl)-thiazol-4-one

The title compound is prepared in accordance with the procedures described herein.

### Example 51

### 5-(4-Fluorobenzyl)-2-(1-pyridin-2-yl-ethyl)thiazol-4-one

The title compound is prepared in accordance with the procedures described herein.

### Example 52

### 2-[4-Oxo-2-(1-p-tolylethyl)-4,5-dihydro-thiazol-5-yl]-N-p-tolylacetamide

The title compound is prepared in accordance with the procedures described herein.

### Example 53

### 2-Phenyl-5-(3-trifluoromethylbenzyl)thiazol-4-one

The title compound is prepared in accordance with the procedures described herein.

### Example 54

### 5-(4-Fluorobenzyl)-2-pyridin-2-yl-thiazol-4-one

The title compound is prepared in accordance with the procedures described herein.

### Example 55

### 2-(4-Oxo-2-phenyl-4,5-dihydrothiazol-5-yl)-N-p-tolylacetamide

The title compound is prepared in accordance with the procedures described herein.

### Example 56

### 2-p-Tolylimino-5-[1-(3-trifluoromethylphenyl)ethyl]-thiazolidin-4-one

The title compound is prepared in accordance with the procedures described herein.

### Example 57

### 5-[1-(4-Fluorophenyl)ethyl]-2-(pyridin-2-ylimino)thiazolidin-4-one

The title compound is prepared in accordance with the procedures described herein.

### Example 58

### 5-[1-Methyl-1-(3-trifluoromethylphenyl)ethyl]-2-p-tolyliminothiazolidin-4-one

The title compound is prepared in accordance with the procedures described herein.

### Example 59

### 5-[1-(4-Fluorophenyl)-1-methylethyl]-2-(pyridin-2-ylimino)thiazolidin-4-one

The title compound is prepared in accordance with the procedures described herein.

### Example 60

### 5-(4-Methoxyphenethyl)-2-(p-tolylimino)thiazolidin-4-one

### (a) Ethyl 2-hydroxy-4-(4-methoxyphenyl)-4-oxobutanoate

Ethyl glyoxylate (50% in toluene, 6 mL, 29.39 mmol) and 4-methoxy acetophenone (4400 mg, 29.39 mmol) were stirred at 135° C. in an open flask for 20 h. The crude reaction mixture was purified by silica gel column chromatography using toluene:EtOAc 2:1 as eluent yielding the title compound as a thick yellowish oil which solidified upon standing (4000 mg, 54%). ¹H NMR: δCDCl₃): 1.40 (t, 3H), 3.45 (dt, 2H), 3.90 (s, 3H), 4.25 (q, 2H), 4.65 (t, 1H), 6.95 (d, 2H), 7.95 (d, 2H).

### (b) Ethyl 2-hydroxy-4-(4-methoxyphenl)butanoate

To a solution of ethyl 2-hydroxy-4-(4-methoxyphenyl)-4-oxobutanoate (500 mg, 1.98 mmol; see step (a) above) in ethanolic HCl (1M, 20 mL), 10% Pd/C (40 mg) was added. The reaction mixture was flushed with H₂ gas and hydrogenated for 6 hours at 1 atm. using a balloon filled with H₂ gas. After stirring for 6 h, the palladium catalyst was filtered off and the solvent and HCl were evaporated yielding the sub-title compound (470 mg, 100%) that was used without purification. ¹H NMR: δ(CDCl₃): 1.30 (t, 3H), 1.95 (m, 1H), 2.10 (m, 1H), 2.75 (m, 2H), 3.80 (s, 3H), 4.25 (q, 2H), 6.85 (d, 2H), 7.15 (d, 2H).

### (c) 1-(Ethoxycarbonyl)-3-(4-methoxyphenyl)propyl 4-methylbenzenesulfonate

To a solution of ethyl 2-hydroxy-4-(4-methoxyphenyl)butanoate (470 mg, 2.0 mmol; see step (b) above) in pyridine (5 mL), tosyl chloride (497 mg, 2.6 mmol) was added in portions at room temperature. The reaction mixture was stirred overnight, diluted with toluene and washed with water (3×). The organic phase was dried (MgSO₄) and concentrated, and the crude product was purified by silica gel chromatography using toluene:EtOAc 20:1 as eluent affording the sub-title compound as a reddish oil (322 mg, 41%). ¹H NMR: δ(CDCl₃): 1.20 (t, 3H), 2.15 (m, 1H), 2.45 (s, 3H), 2.55-2.70 (m, 2H), 8.85 (S, 3H), 4.15 (t, 2H), 5.90 (m, 1H), 6.85 (d, 2H), 7.10 (d, 2H), 7.40 (d, 2H), 7.90 (d, 2H).

### (d) 5-(4-Methoxyphenethyl)-2-(p-tolylimino)thiazolidin-4-one

1-(Ethoxycarbonyl)-3-(4-methoxyphenyl)propyl 4-methylbenzenesulfonate (155 mg, 0.40 mmol; see step (c) above), p-tolyl thiourea (67 mg, 0.40 mmol) and NaOAc (36 mg, 0.44 mmol) were dissolved in 1.0 mL 95% EtOH. The reaction mixture was refluxed for 16 h, concentrated in vacuum and partitioned between EtOAc and water. After three extractions with EtOAc, the combined organic phases were dried (MgSO₄) and concentrated, and the crude product was purified by silica gel column chromatography using toluene:EtOAc 2:1 as eluent. Further purification by recrystilization from hot MeOH yielded the title compound as a beige-brown powder (42 mg, 31%). LC-MS (A) t_{R}: 8.50 min. ES-MS m/z: 341.2 (MH+). ¹H NMR: δ(DMSO-d₆): 1.80-2.00 (m, 1H), 2.20-2.40 (s, 3H overlap with m, 1H), 2.60 (m, 1H), 2.75 (m, 1H), 3.70 (s, 3H), 4.15-4.25 (m, 1H), 6.80-6.90 (m, 2H), 6.95 (m, 1H), 7.05-7.20 (m, 4H), 7.60 (d, 1H).

### Example 61

### 5-(4-Methoxyphenethyl)-2-(phenylimino)thiazolidin-4-one

The title compound was prepared in accordance with Example 60, purified by flash chromatography and recrystallised from hot methanol to give 35 mg of the title compound as an off-white powder. LC-MS (A) t_{R}: 8.58 min. ES-MS m/z: 327.0 (MH+). ¹H NMR: δ(DMSO-d₆): 1.95 (m, 1H), 2.20-2.40 (m, 1H), 2.65 (m, 1H), 2.70 (m, 1H), 3.70 (s, 3H), 4.25 (m, 1H), 6.85 (m, 2H), 6.95-7.20 (m, 4H), 7.40 (m, 2H), 7.70 (d, 1H).

### Example 62

### 2-(p-Tolylimino)-5-phenethylthiazolidin-4-one

The title compound was prepared in accordance with Example 60, purified by flash chromatography and recrystallised from hot methanol to give 96 mg of the title compound. LC-MS (B) t_{R}: 1.75 min, m/z 310.9 (MH+). ¹H NMR: δ(DMSO-d₆): 2.00 (m, 1H), 2.30 (s, 3H), 2.36 (m, 1H), 2.61 (m, 1H), 2.75 (m, 1H), 4.21 (dm, 1H), 6.91 (m, 1H), 7.19 (m, 5H), 7.29 (m, 2H), 7.58 (d, 2H).

### Example 63

### 2-p-Tolylimino-5-[2-(3-trifluoromethyl-phenyl)-ethyl]-thiazolidin-4-one

The title compound is prepared in accordance with the procedures described herein.

### Example 64

### 5-[2-(4-Fluorophenyl)-ethyl]-2-(pyridin-2-ylimino)-thiazolidin-4-one

The title compound is prepared in accordance with the procedures described herein.

### Example 65

### 2-(p-Tolylimino)-5-(3-phenylpropyl)thiazolidin-4-one

The following procedure is analogous to that described in Example 26 above.

### (a) 2-(p-Tolylimino)thiazolidin-4-one

A mixture of ethyl 2-bromoacetate (0.25 mL, 2.29 mmol), N-(4-methylphenyl)thiourea (381 mg, 2.29 mmol) and sodium acetate (212 mg, 2.54 mmol) in ethanol (5 mL) was refluxed overnight. The mixture was concentrated, diluted with dichloromethane and washed with water. The organic layer was dried with MgSO₄, filtered and concentrated. The crude product was purified by silica gel chromatography using toluene:ethyl acetate (2:1) as eluent (441 mg) and recrystallised from methanol to give 178 mg (0.86 mmol, 38%) of the sub-title compound. LC-MS (A) t_{R}: 4.68 min, m/z 207.2 (MH+). ¹H NMR: δ(DMSO-d₆): 2.26 (s, 3H), 3.84 (d, 2H), 6.69 (d, 1H), 7.16 (d, 2H), 7.57 (d, 1H).

### (b) 2-(p-Tolylimino)-5-(3-phenylpropylidene)thiazolidin-4-one

A mixture of 2-(p-tolylimino)thiazolidin-4-one (100 mg, 0.48 mmol; see step (a) above), 3-phenyl propionaldehyde (72 mg, 0.73 mmol) and NaOAc (62 mg, 0.75 mmol) in 2 mL glacial AcOH was refluxed for 21 h. The solvent was evaporated, and the crude product was purified by silica gel column chromatography using toluene:acetone 3:1 as eluent yielding 120 mg (78%) of the sub-title compound as a brown powder. LC-MS (A) t_{R}: 9.30 min. ES-MS m/z: 323 (MH+).

### (c) 2-(p-Tolylimino)-5-(3-phenylpropyl)thiazolidin-4-one

To a solution of 2-(p-tolylimino)-5-(3-phenylpropylidene)thiazolidin-4-one (220 mg, 0.68 mmol; see step (b) above) in pyridine (0.55 mL) and THF (0.50 mL), LiBH₄ (2M in THF, 0.75 mL, 1.50 mmol) was slowly added at room temperature, and the resulting mixture was refluxed for 5 h. The mixture was allowed to attain room temperature, and the reaction was quenched by addition of 1M HCl. Water was added and the mixture extracted three times with EtOAc. The combined organic phases were dried with MgSO₄, filtered and concentrated. The crude product was purified by silica gel chromatography using toluene:EtOAc 2:1 as eluent yielding 23 mg (10%) of the title compound. LC-MS (A) t_{R}: 9.14 min. ES-MS m/z: 325.4 (MH+).

### Example 66

### 2-p-Tolylimino-5-[3-(3-trifluoromethylphenyl)propyl]thiazolidin-4-one

The title compound is prepared in accordance with the procedures described herein.

### Example 67

### 5-[3-(4-Fluorophenyl)propyl]-2-(pyridin-2-ylimino)thiazolidin-4-one

The title compound is prepared in accordance with the procedures described herein.

### Example 68

### 5-(3-Phenylallylidene)-2-(phenylimino)thiazolidin-4-one

A solution of 2-(phenylimino)thiazolidin-4-one (100 mg, 0.52 mmol), cinnamyl aldehyde (171 mg, 0.78 mmol) and NaOAc (66 mg, 0.80 mmol) in 2 mL glacial AcOH was refluxed for 18 h, while the product precipitated. The suspension was allowed to attain room temperature, diluted with 2 mL of AcOH, transferred to a tube and centrifuged. The mother liquid was removed and an additional 4 mL of AcOH was added, and the tube was again centrifuged. This washing procedure was repeated with 2×4 mL of toluene. The residue was dried in vacuo yielding the title compound (135 mg, 85%) as a yellow powder. LC-MS (A) t_{R}: 9.46 min. ES-MS m/z: 307.0 (MH+).

### Example 69

### 2-p-Tolylimino-5-[(3-trifluoromethylphenylamino)methyl]thiazolidin-4-one

The title compound is prepared in accordance with the procedures described herein.

### Example 70

### 5-[(4-Fluorophenylamino)methyl]-2-(pyridin-2-ylimino)thiazolidin-4-one

The title compound is prepared in accordance with the procedures described herein.

### Example 71

### 5-{[Methyl-(3-trifluoromethylphenyl)amino]methyl}-2-p-tolylimino-thiazolidin-4-one

The title compound is prepared in accordance with the procedures described herein.

### Example 72

### 5-{[(4-Fluorophenyl)methylamino]methyl}-2-(pyridin-2-ylimino)thiazolidin-4-one

The title compound is prepared in accordance with the procedures described herein.

### Example 73

### 2-p-Tolylimino-5-(3-trifluoromethyl-phenoxymethyl)-thiazolidin-4-one

The title compound is prepared in accordance with the procedures described herein.

### Example 74

### 5-(4-Fluorophenoxymethyl)-2-(pyridin-2-ylimino)thiazolidin-4-one

The title compound is prepared in accordance with the procedures described herein.

### Example 75

### 2-p-Tolylimino-5-(3-trifluoromethylphenylsulfanylmethyl)thiazolidin-4-one

The title compound is prepared in accordance with the procedures described herein.

### Example 76

### 5-(4-Fluorophenylsulfanylmethyl-2-(pyridin-2-ylimino)thiazolidin-4-one

The title compound is prepared in accordance with the procedures described herein.

### Example 77

### 2-p-Tolylimino-5-[(3-trifluoromethylbenzylamino)methyl]thiazolidin-4-one

The title compound is prepared in accordance with the procedures described herein.

### Example 78

### 5-[(4-Fluorobenzylamino)methyl]-2-(pyridin-2-ylimino)thiazolidin-4-one

The title compound is prepared in accordance with the procedures described herein.

### Example 79

### 5-{[Methyl-(3-trifluoromethylbenzyl)amino]methyl}-2-p-tolylimino-thiazolidin-4-one

The title compound is prepared in accordance with the procedures described herein.

### Example 80

### 5-{[(4-Fluorobenzyl)methylamino]methyl}-2-(pyridin-2-ylimino)thiazolidin-4-one

The title compound is prepared in accordance with the procedures described herein.

### Example 81

### N-(4-Oxo-2-p-tolylimino-thiazolidin-5-ylmethyl)-3-trifluoromethyl-benzamide

The title compound is prepared in accordance with the procedures described herein.

### Example 82

### 4-Fluoro-N-[4-oxo-2-(pyridin-2-ylimino)thiazolidin-5-ylmethyl]benzamide

The title compound is prepared in accordance with the procedures described herein.

### Example 83

### N-Methyl-N-(4-oxo-2-p-tolylimino-thiazolidin-5-ylmethyl)-3-trifluoromethyl-benzamide

The title compound is prepared in accordance with the procedures described herein.

### Example 84

### 4-Fluoro-N-methyl-N-[4-oxo-2-(pyridin-2-ylimino)thiazolidin-5-ylmethyl]-benzamide

The title compound is prepared in accordance with the procedures described herein.

### Example 85

### N-(4-Oxo-2-p-tolylimino-thiazolidin-5-ylmethyl)-2-(3-trifluoromethyl-phenyl)-acetamide

The title compound is prepared in accordance with the procedures described herein.

### Example 86

### 2-(4-Fluorophenyl)-N-[4-oxo-2-(pyridin-2-ylimino)thiazolidin-5-ylmethyl]-acetamide

The title compound is prepared in accordance with the procedures described herein.

### Example 87

### 1-(4-Oxo-2-p-tolyliminothiazolidin-5-ylmethyl)-3-(3-trifluoromethylphenyl)urea

The title compound is prepared in accordance with the procedures described herein.

### Example 88

### 1-(4-Fluorophenyl)-3-[4-oxo-2-(pyridin-2-ylimino)thiazolidin-5-ylmethyl]urea

The title compound is prepared in accordance with the procedures described herein.

### Example 89

### (4-Oxo-2-p-tolyliminothiazolidin-5-ylmethyl)-carbamic Acid 3-trifluoromethyl-phenyl Ester

The title compound is prepared in accordance with the procedures described herein.

### Example 90

### [4-Oxo-2-(pyridin-2-ylimino)thiazolidin-5-ylmethyl]carbamic acid 4-fluorophenyl Ester

The title compound is prepared in accordance with the procedures described herein.

### Example 91

### (3-Trifluoromethylphenyl)carbamic Acid 4-oxo-2-p-tolyliminothiazolidin-5-ylmethyl Ester

The title compound is prepared in accordance with the procedures described herein.

### Example 92

### (4-Fluorophenyl)carbamic Acid 4-oxo-2-(pyridin-2-ylimino)thiazolidin-5-ylmethyl Ester

The title compound is prepared in accordance with the procedures described herein.

### Example 93

5-(4-Chlorobenzyl)-2-(pyridin-2-ylimino)thiazolidin-4-one

### Example 94

5-(4-Methoxbenzyl)-2-(pyridin-2-ylimino)thiazolidin-4-one

### Example 95

5-(4-Fluorobenzyl)-2-(pyridin-2-ylimino)thiazolidin-4-one

### Example 96

5-(2-Methylbenzyl)-2-(pyridin-2-ylimino)thiazolidin-4-one

### Example 97

5-(4-Methylbenzyl)-2-(pyridin-2-ylimino)thiazolidin-4-one

### Example 98

5-(2,3-Dichlorobenzyl)-2-(pyridin-2-ylimino)thiazolidin-4-one

### Example 99

5-(4-Bromobenzyl)-2-(pyridin-2-ylimino)thiazolidin-4-one

### Example 100

### 5-(3-(Trifluoromethyl)benzyl)-2-(pyridin-2-ylimino)thiazolidin-4-one

The title compound was prepared in accordance with Example 4, purified by flash chromatography and recrystallised from hot methanol yielding 94 mg of the title compound. LC-MS (B) t_{R}: 0.73 min, m/z 352.4 (MH+). ¹H NMR: δ(DMSO-d₆): 3.15 (m, 1H), 3.45 (dd, 1H), 4.60 (m, 1H), 7.19 (m, 2H), 7.5-7.6 (m, 4H), 7.78 (m, 1H), 8.30 (m, 1H).

### Example 101

### 5-(4-Fluorobenzyl)-2-(benzylamino)thiazol-4(5H)-one

The title compound was prepared in accordance with Example 4, purified by flash chromatography and recrystallised from hot methanol yielding 322 mg of the title compound. LC-MS (B) t_{R}: 1.45 min, m/z 315.1 (MH+). ¹H NMR: δ(DMSO-d₆): 2.95 (dd, 1H), 3.30 (m, 1H, HDO), 4.48-4.62 (m, 3H), 7.05-7.33 (m, 9H).

### Example 102

### 5-(3-(Trifluoromethyl)benzyl)-2-(benzylimino)thiazolidin-4-one

The title compound was prepared in accordance with Example 4, purified by flash chromatography and recrystallised from hot methanol yielding 133 mg of the title compound. LC-MS (A) t_{R}: 6.08 min, m/z 365.4 (MH+). ¹H NMR: δ(DMSO-d₆): 3.11 (dd, 1H), 3.42 (dd, 1H), 4.50 (d, 1H), 4.59 (d, 1H), 4.69 (dd, 1H), 7.13 (d, 2H), 7.29 (m, 4H), 7.5-7.6 (m, 4H).

### Example 103

### 2-((Pyridin-2-yl)methylamino)-5-(4-fluorobenzyl)thiazol-4(5H)-one

The title compound is prepared in accordance with the procedures described herein.

### Example 104

### N-(5-(3-(Trifluoromethyl)benzyl)-4-oxothiazolidin-2-ylidene)benzamide

To a suspension of 5-(3-(trifluoromethyl)benzyl)-2-aminothiazol-4(5H)-one (100 mg, 0.36 mmol, prepared in accordance with the procedures described in Example 4) and triethylamine (76 uL, 0.55 mmol) in CH₂Cl₂ (3 ml), benzoyl chloride (50 uL, 0.40 mmol) was dropwise added. The reaction mixture was stirred at room temperature overnight and poured into a saturated solution of NaHCO₃ in water. The water phase was extracted with CH₂Cl₂, and the organic phase was dried with MgSO₄, filtered and concentrated in vacuum. The crude material was purified by column chromatography using a gradient of CH₂Cl₂/MeOH (0-1%) as eluent to give 38 mg (0.10 mmol, 28%) of the title compound as colourless oil. Recrystallisation from CH₂Cl₂/iso-hexane gave 22 mg of the title compound as white solid. LC-MS (A) t_{R}: 8.72 min. ES-MS m/z 379.0 (MH+). ¹H NMR: δ (400 MHz) (CDCl₃): 3.23 (dd, 1H), 3.64 (dd, 1H), 4.34 (dd, 1H), 7.46-7.61 (m, 7H), 8.12 (d, 2H) ppm.

### Example 105

### N-(5-(3-(Trifluoromethyl)benzyl)-4-oxothiazolidin-2-ylidene)-4-chlorobenzamide

The title compound was prepared in accordance with Example 104, purified by flash chromatography (83 mg, colourless oil) and recrystallised from CH₂Cl₂/iso-hexane to give 72 mg of the title compound as white solid. LC-MS (A) t_{R}: 8.92 min. ES-MS m/z 413.2 (MH+). ¹H NMR: δ (400 MHz) (CDCl₃): 3.22 (dd, 1H), 3.61 (dd, 1H), 4.34 (dd, 1H), 7.42-7.49 (m, 4H), 7.52-7.59 (m, 2H), 8.12 (d, 2H) ppm.

### Example 106

### N-(5-(3-(Trifluoromethyl)benzyl)-4-oxothiazolidin-2-ylidene)-4-methylbenzamide

The title compound was prepared in accordance with Example 104, purified by flash chromatography (32 mg, colourless oil) and recrystallised from CH₂Cl₂/iso-hexane to give 10 mg of the title compound as white solid. LC-MS (A) t_{R}: 8.73 min. ES-MS m/z 393.0 (MH+). ¹H NMR: δ (400 MHz) (CDCl₃): 2.54 (s, 3H), 3.30 (dd, 1H), 3.74 (dd, 1H), 4.41 (dd, 1H), 7.35-7.42 (m, 2H), 7.52-7.71 (m, 3H), 7.78 (d, 1H), 8.12 (d, 2H) ppm.

### Example 107

### N-(5-(4-Fluorobenzyl)-4,5-dihydro-4-oxothiazol-2-yl)picolinamide

The title compound is prepared in accordance with the procedures described herein.

### Example 108

### Phenyl 5-(3-(trifluoromethyl)benzyl)-4-oxothiazolidin-2-ylidenecarbamate

The title compound was prepared in accordance with Example 104, purified by flash chromatography (88 mg, colourless oil) and recrystallised from CH₂Cl₂/iso-hexane to give 74 mg of the title compound as white solid. LC-MS (A) t_{R}: 8.73 min. ES-MS m/z 395.0 (MH+). ¹H NMR: δ (400 MHz) (CDCl₃): 3.22 (dd, 1H), 3.61 (dd, 1H), 4.37 (dd, 1H), 7.21-7.28 (m, 3H), 7.37-7.58 (m, 6H) ppm.

### Example 109

### Pyridin-2-yl 5-(4-fluorobenzyl)-4.5-dihydro-4-oxothiazol-2-ylcarbamate

The title compound is prepared in accordance with the procedures described herein.

### Example 110

### 1-(5-(3-(Trifluoromethyl)benzyl)-4-oxothiazolidin-2-ylidene)-3-phenylurea

5-(3-(Trifluoromethyl)benzyl)-2-aminothiazol-4(5H)-one (100 mg, 0.36 mmol, prepared in accordance with Example 4) was dissolved in toluene (3 mL), and phenyl isocyanate (44 uL, 0.40 mmol) was added dropwise. The reaction mixture was heated at reflux for 3 hours. The precipitate that had formed was filtered off, washed with toluene and dried in vacuum to give 137 mg (0.35 mmol, 97%) of the title compound as a white solid. ¹H NMR: δ (400 MHz) (DMSO-d₆): 3.21 (dd, 1H), 3.46 (dd, 1H), 4.64 (dd, 1H), 6.98-7.02 (m, 1H), 7.23-7.28 (m, 2H), 7.56-7.68 (m, 6H), 9.79 (br.s, 1H) ppm.

### Example 111

### 1-(5-(3-(Trifluoromethyl)benzyl)-4-oxothiazolidin-2-ylidene)-3-p-tolylurea

The title compound was prepared in accordance with Example 110, yielding 126 mg of the title compound as a white solid. ¹H NMR: δ (400 MHz) (DMSO-d₆): 2.20 (s, 3H), 3.21 (dd, 1H), 3.46 (dd, 1H), 4.63 (dd, 1H), 7.04 (d, 2H), 7.44-7.66 (m, 6H), 9.71 (br.s, 1H) ppm.

### Example 112

### 1-(5-(3-(Trifluoromethyl)benzyl)-4-oxothiazolidin-2-ylidene)-3-(4-chlorophenyl)-urea

The title compound was prepared in accordance with Example 110, yielding 161 mg of the title compound as a white solid. ¹H NMR: δ (400 MHz) (DMSO-d₆): 3.19 (dd, 1H), 3.43 (dd, 1H), 4.64 (dd, 1H), 7.28 (d, 2H), 7.58-7.69 (m, 6H), 9.95 (br.s, 1H) ppm.

### Example 113

### 1-(5-(4-Fluorobenzyl)-4.5-dihydro-4-oxothiazol-2-yl)-3-(pyridin-2-yl)urea

The title compound is prepared in accordance with the procedures described herein.

### Example 114

### 5-(3-(Trifluoromethyl)benzyl)-2-tosyliminothiazolidin-4-one

5-(3-(Trifluoromethyl)benzyl)-2-aminothiazol-4(5H)-one (100 mg, 0.36 mmol, prepared in accordance with Example 4) was dissolved in pyridine (3 mL), and tosyl chloride (77 mg, 0.40 mmol) was added. The reaction mixture Was stirred at room temperature overnight and poured into a saturated solution of NaHCO₃ in water. The water phase was extracted with CH₂Cl₂, and the organic phase was dried with MgSO₄, filtered and concentrated in vacuum. The crude material was purified by column chromatography using a gradient of CH₂Cl₂/MeOH (0-1%) as eluent to give 55 mg (0.13 mmol, 36%) of the title compound as colourless oil. Recrystallisation from CH₂Cl₂/iso-hexane yielded 34 mg of a white solid. LC-MS (A) t_{R}: 8.53 min. ES-MS m/z 429.2 (MH+). ¹H NMR: δ (400 MHz) (CDCl₃): 2.44 (s, 3H), 3.22 (dd, 1H), 3.58 (dd, 1H), 4.40 (dd, 1H), 7.33 (d, 2H), 7.42-7.51 (m, 3H), 7.58 (d, 1H), 7.78 (d, 2H) ppm.

### Example 115

### 5-(3-(Trifluoromethyl)benzyl)-2-phenylsulfonyliminothiazolidin-4-one

The title compound was prepared in accordance with Example 114, purified by flash chromatography (49 mg, colourless oil) and recrystallised from CH₂Cl₂/iso-hexane to give 29 mg of the title compound as a white solid. LC-MS (A) t_{R}: 8.37 min. ES-MS m/z 415.0 (MH+). ¹H NMR: δ (400 MHz) (CDCl₃): 3.24 (dd, 1H), 3.57 (dd, 1H), 4.40 (dd, 1H), 7.44-7.67 (m, 7H), 7.91 (d, 2H) ppm.

### Example 116

### 5-(3-(Trifluoromethyl)benzyl)-2-(4-chlorophenyl)sulfonyliminothiazolidin-4-one

The title compound was prepared in accordance with Example 114, purified by flash chromatography (43 mg, colourless oil) and recrystallised from CH₂Cl₂/iso-hexane to give 20 mg of the title compound as a white solid. LC-MS (A) t_{R}: 8.78 min. ES-MS m/z 449.2 (MH+). ¹H NMR: δ (400 MHz) (CDCl₃): 3.35 (dd, 1H), 3.57 (dd, 1H), 4.40 (dd, 1H), 7.41-7.45 (m, 5H), 7.59 (d, 1H), 7.83 (d, 2H) ppm.

### Example 117

### 5-(4-Fluorobenzyl)-2-(2-pyridylsulfonylamino)thiazol-4(5H)-one

The title compound is prepared in accordance with the procedures described herein.

### Example 118

### 5-(3-(Trifluoromethyl)benzyl)-2-(isopropylamino)thiazol-4(5H)-one

The title compound was prepared in accordance with Example 4, purified by flash chromatography and preparative HPLC to give 170 mg of the title compound as an off-white powder. LC-MS (A) t_{R}: 7.08 min. ES-MS m/z: 317.0 (MH+). ¹H NMR: δ(DMSO-d₆): 1.05 (d, 3H), 1.15 (d, 3H), 3.10 (dd, 1H), 3.45 (dd, 1H), 4.00 (m, 1H), 4.65 (dd, 1H), 7.50-7.65 (m, 4H), 9.00 (d, 1H).

### Example 119

### 5-(3-(Trifluoromethyl)benzyl)-2-(cyclohexylamino)thiazol-4(5H)-one

The title compound was prepared in accordance with Example 4, purified by flash chromatography and preparative HPLC to give 120 mg of the title compound as an off-white powder. LC-MS (A) t_{R} 9.08 min. ES-MS m/z 357.2 (MH+). ¹H NMR: δ(DMSO-d₆): 1.00-1.40 (m, 5H), 1.54 (d, 1H), 1.60-1.90 (m, 4H), 3.05 (dd, 1H), 3.40 (dd, 1H), 3.65 (m, 1H), 4.55 (dd, 1H), 7.45-7.65 (m, 4H), 9.05 (d, 1H).

### Example 120

### 5-(3-(Trifluoromethyl)benzyl)-2-(methylamino)thiazol-4(5H)-one

The title compound was prepared in accordance with Example 4 and purified by flash chromatography to give 240 mg of the title compound as an oil. LC-MS (A) t_{R}: 4.74 min, m/z 289.2 (MH+).

### Example 121

### 2-(p-Tolylimino)-5-methylthiazolidin-4-one

The title compound was prepared in accordance with Example 4, purified by flash chromatography and recrystallised from methanol to give 149 mg of the title compound. LC-MS (A) t_{R}: 5.57 min, m/z 221.2 (MH+). ¹H NMR: δ(DMSO-d₆): 1.47 (dd, 3H), 2.25 (s, 3H), 3.50 (dd, 1H), 4.23 (q, 1H), 6.89 (t, 1H), 6.88 (d, 1H), 7.16 (m, 2H), 7.57 (d, 1H).

### Example 122

### 2-(p-Tolylimino)thiazolidin-4-one

The title compound was prepared in accordance with Example 4, purified by flash chromatography and recrystallised from methanol to give 178 mg of the title compound. LC-MS (A) t_{R}: 4.68 min, m/z 207.2 (MH+). ¹H NMR: δ(DMSO-d₆): 2.26 (s, 3H), 3.84 (d, 2H), 6.69 (d, 1H), 7.16 (d, 2H); 7.57 (d, 1H).

### Example 123

### 5-(3-(Trifluoromethyl)benzyl)-2-aminothiazol-4(5H)-one

The title compound was prepared in accordance with Example 4. The reaction mixture was concentrated and partitioned between dichloromethane and water. A solid was filtered off to give 1.22 g of the title compound. The organic layer was dried (MgSO₄) and concentrated, and the residue was triturated with iso-hexane to yield another 1.02 g of the title compound (2.24 g in total). LC-MS (A) t_{R}: 5.3 min, m/z 275.2 (MH+). ¹H NMR: δ(DMSO-d₆): 3.05 (dd, 1H), 3.45 (dd, 1H), 4.63 (dd, 1H), 7.56 (m, 4H), 8.80 (b, 2H).

### Example 124

2-(2-(4-Carboxyphenylimino)-4-oxothiazolidin-5-yl)-N-(3-methoxyphenyl)-acetamide

### Example 125

2-(2-(4-Hydroxyphenylimino)-4-oxothiazolidin-5-yl)-N-(4-bromophenyl)-acetamide

### Example 126

2-(2-(4-Ethoxyphenylimino)-4-oxothiazolidin-5-yl)-N-(4-bromophenyl)acetamide

### Example 127

2-(2-(3-Hydroxyphenylimino)-4-oxothiazolidin-5-yl)-N-(4-bromophenyl)-acetamide

### Example 128

2-(2-(4-Hydroxyphenylimino)-4-oxothiazolidin-5-yl-N-phenylacetamide

### Example 129

2-(2-(4-Hydroxyphenylimino)-4-oxothiazolidin-5-yl)-N-(4-fluorophenyl)-acetamide

### Example 130

2-(2-(p-Tolylimino)-4-oxothiazolidin-5-yl)-N-p-tolylacetamide

### Example 131

2-(2-(4-Methoxyphenylimino)-4-oxothiazolidin-5-yl)-N-(4-methoxyphenyl)-acetamide

### Example 132

2-(2-(4-Ethoxyphenylimino)-4-oxothiazolidin-5-yl)-N-phenylacetamide

### Example 133

Ethyl 4-(2-(2-(4-ethoxyphenylimino)-4-oxothiazolidin-5-yl)acetamido)benzoate

### Example 134

2-(2-(3-(Trifluoromethyl)phenylimino)-4-oxothiazolidin-5-yl)acetic acid

### Example 135

N-(2,4-Dimethylphenyl)-2-(4-oxo-2-(phenylimino)thiazolidin-5-yl)acetamide

### Example 136

N-(2,4-Dimethoxyphenyl)-2-(4-oxo-2-(phenylimino)thiazolidin-5-yl)acetamide

### Example 137

2-(4-Oxo-2-(4-sulfonylamidophenylimino)thiazolidin-5-yl-N-p-tolylacetamide

### Example 138

N-(4-Fluorophenyl)-2-(4-oxo-2-(4-oxo-2-(phenylimino)thiazolidin-5-yl)acetamide

### Example 139

2-(2-(m-Tolylimino)-4-oxothiazolidin-5-yl)-N-(2-chlorophenyl)acetamide

### Example 140

2-(2-(2,5-Dimethylphenylimino)-4-oxothiazolidin-5-yl)-N-(2,4-dichlorophenyl)-acetamide

### Example 141

2-(4-Oxo-3-phenyl-2-(phenylimino)thiazolidin-5-yl)-N-p-tolylacetamide

### Example 142

2-(2-(Cyclohexylimino)-4-oxothiazolidin-5-yl)-N-phenylacetamide

### Example 143

2-(2-(Methylimino)-4-oxothiazolidin-5-yl)-N-(2,4-dimethylphenyl)acetamide

### Example 144

N-Ethyl-2-(2-(methylimino)-4-oxothiazolidin-5-yl)acetamide

### Example 145

2-(2-(Allylimino)-4-oxothiazolidin-5-yl)-N-(2-nitrophenyl)acetamide

### Example 146

### 1,1-Dioxo-1λ⁶-[1,4,2]dithiazolidin-3-ylidene]-p-tolyl-amine

### (a) 2-chloromethanesulfonamide

Ammonia gas was bubbled through a solution of chloromethanesulfonyl chloride (5.0 g, 34 mmol) in Et₂O (50 mL) at 0° C. The reaction mixture was stirred at ambient temperature for 2 hours. The precipitate (ammonium chloride) was filtered off and washed with EtOAc (3×). The combined organic phases were dried (Na₂SO₄) and concentrated to give 2.96 g (67%) of the crude sub-title compound as a white solid. The compound was used without further purification. ¹H NMR: δ(DMSO-d₆): 5.74 (s, 2H), 7.33 (s, 2H).

### (b) 1,1-Dioxo-1λ⁶-[1,4,2]dithiazolidin-3-ylidene]-p-tolyl-amine

An aqueous solution of NaOH (18 M, 1.38 mL, 25 mmol) was added over 30 minutes to a solution of crude 2-chloromethanesulfonamide (2.96 g, ˜23 mmol) and 4-methylphenyl isothiocyanate (3.75 g, 24.0 mmol) in acetone (14 mL) at 50° C. The resulting mixture was stirred overnight at ambient temperature. The reaction mixture was acidified with hydrochloric acid (1 M), and the organic solvent was evaporated in vacuo. Water and EtOAc was added, and the water phase was extracted with EtOAc (×3). The combined organic phases were dried (Na₂SO₄) and concentrated, and the crude product was purified by silica gel column chromatography (toluene/EtOAc 4:1 to 2:1) to give 3.46 g (63%) of the title compound as a white solid. LC-MS (A) t_{R}: 7.70 min. ES-MS m/z: 243.0 (MH+). ¹H NMR: δ(DMSO-d₆): 2.28 (s, 3H), 4.75 (s, 2H), 7.22 (d, 2H), 7.45 (d, 2H).

### Example 147

### [1,1-Dioxo-5-(3-(trifluoromethyl)phenyl)(hydroxy)methyl)-1λ⁶-[1,4,2]dithiazo-idin-3-ylidene]-p-tolyl-amine

LDA (1.8M, 2.1 mL, 3.72 mmol) was added over 20 minutes to a solution of 1,1-Dioxo-1λ⁶-[1,4,2]dithiazolidin-3-ylidene]-p-tolyl-amine (300 mg, 1.24 mmol) in dry THF (2 mL) at 0° C. under nitrogen atmosphere. The reaction mixture was allowed to reach room temperature within 1 hour and stirred at RT for an additional 3 hours. After re-cooling the reaction mixture to 0° C., a solution of 3-(trifluoromethyl)benzaldehyde (420 μL, 3.1 mmol) in dry THF (0.5 mL) was added dropwise. The reaction temperature was allowed to slowly reach room temperature, and the resulting mixture was left overnight. Hydrochloric acid and EtOAc were added, and the water phase was extracted with EtOAc (×3). The combined organic phases were dried (Na₂SO₄) and the solvent was removed in vacuo. The crude product was purified by silica gel column chromatography (toluene/EtOAc 100:0 to 2:1) to give 364 mg (70%) of the title compound as a 1:1 mixture of diastereoisomers. LC-MS (A) t_{R}: 10.02 min. ES-MS m/z: 417.2 (MH+). ¹H NMR (1:1 diastereomeric mixture): δ(CD₃CN-d₃): 2.31 (s, 3H), 2.34 (s, 3H), 5.13 (m, 2H), 5.27 (d, 1H), 5.55 (d, 1H), 7.19 (d, 2H), 7.22 (d, 2H), 7.31 (m, 2H), 7.40 (m, 2H), 7.58 (m, 2H), 7.66 (m, 2H), 7.74 (m, 2H), 7.81 (m, 2H).

### Example 148

### [1,1-Dioxo-5-(3-(trifluoromethyl)benzylidene)-1λ⁶-[1,4,2]dithiazolidin-3-ylid-ene]-p-tolyl-amine

Trifluoroacetic anhydride (136 μL, 0.99 mmol) was added to a solution of the compound of Example 147 (370 mg, 0.89 mmol), 4-(dimethylamino)pyridine (27 mg, 0.22 mmol) and Et₃N (370 μL, 2.67 mmol) in DCM (2.5 mL) at 0° C. under nitrogen atmosphere. The reaction mixture was stirred at ambient temperature for 3 hours. Hydrochloric acid (1 M) and EtOAc was added, and the water phase was extracted with EtOAc (×3). The combined organic phases were dried (Na₂SO₄) and concentrated, and the crude product was purified by silica gel column chromatography (toluene/EtOAc 100:0 to 2:1) to give 293 mg (84%) of the title compound as a pale white solid. LC-MS (A) t_{R}: 9.57 min. ES-MS m/z: 399.2 (MH+). ¹H NMR: δ(DMSO-d₆): 2.33 (s, 3H), 7.28 (d, 2H), 7.53 (d, 2H), 7.86 (m, 4H), 7.92 (s, 1H).

### Example 149

### [1,1-Dioxo-5-(3-trifluoromethylbenzyl)-1λ^{6[}1,4,2]dithiazolidin-3-ylidene]-p-tolylamine

The title compound is prepared in accordance with the procedures described herein.

### Example 150

### [1,1-Dioxo-5-(4-(fluoro)phenyl)(hydroxy)methyl)-1λ⁶-[1,4,2]dithiazolidin-3-ylidene]-p-tolyl-amine

The title compound was prepared in accordance with the procedures described in Examples 146 and 147, and purified by flash chromatography to give 312 mg of the title compound as a 1:1 mixture of diastereoisomers. LC-MS (A) t_{R}: 9.10 min. ES-MS m/z: 367.2 (MH+). ¹H NMR (1:1 diastereomeric mixture): δ(CD₃CN-d₃): 5.09 (m, 2H), 5.21 (d, 1H), 5.39 (d, 1H), 7.13 (m, 4H), 7.20 (m, 4H), 7.38-7.45 (m, 4H), 7.54 (m, 4H).

### Example 151

### [1,1-Dioxo-5-(4-(fluoro)benzylidene)-1λ⁶-[1,4,2]dithiazolidin-3-ylidene]-p-tolyl-amine

The title compound was prepared in accordance with the procedures described in Examples 146 to 148, and purified by flash chromatography to give 176 mg of the title compound as a pale white solid. LC-MS (A) t_{R}: 10.14 min. ES-MS m/z: 349.4 (MH+). ¹H NMR: δDMSO-d₆): 2.35 (s, 3H), 7.32 (d, 2H), 7.45 (d, 2H), 7.57 (m, 2H), 7.70 (m, 2H), 7.79 (s, 1H).

### Example 152

### [1,1-Dioxo-5-(3-(trifluoromethyl)phenyl)(hydroxy)methyl-1λ⁶-[1,4,2]dithiazol-idin-3-ylidene]-4-chlorophenyl-amine

The title compound was prepared in accordance with the procedures described in Examples 146 and 147, and purified by flash chromatography to give 0.5 g of the title compound as a 1:1 mixture of diastereoisomers. LC-MS (A) t_{R}: 9.54 min. ES-MS m/z: 437.2 (MH+). ¹H NMR (1:1 diastereomeric mixture): δ(CD₃CN-d₃): 5.28 (m, 2H), 5.40 (d, 1H), 5.68 (d, 1H), 7.51 (m, 4H), 7.60 (d, 2H), 7.71 (m, 2H), 7.80 (m, 2H), 7.58 (m, 2H), 7.85 (m, 2H), 7.96 (m, 2H).

### Example 153

### [5-(4-Fluoro-benzyl)-1,1-dioxo-1λ⁶-[1,4,2]dithiazolidin-3-ylidene]-pyridin-2-yl-amine

The title compound is prepared in accordance with the procedures described herein.

### Example 154

### 2-(1,1-Dioxo-3-n-tolylimino-1λ⁶-[1,4,2]dithiazolidin-5-yl)-N-p-tolyl-acetamide

The title compound is prepared in accordance with the procedures described herein.

### Example 155

### 5-(3-(Trifluoromethyl)benzyl)-4-methyl-N-p-tolylthiazol-2-amine

The title compound is prepared in accordance with the procedures described herein.

### Example 156

### N-(5-(4-Fluorobenzyl)-4-methylthiazol-2-yl)pyridin-2-amine

The title compound is prepared in accordance with the procedures described herein.

### Example 157

### 5-(3-(Trifluoromethyl)benzyl)-4-(trifluoromethyl)-N-p-tolylthiazol-2-amine

The title compound is prepared in accordance with the procedures described herein.

### Example 158

### N-(5-(4-Fluorobenzyl)-4-(trifluoromethyl)thiazol-2-yl)pyridin-2-amine

The title compound is prepared in accordance with the procedures described herein.

### Example 159

### 2-(4-Chlorophenylimino)-5-((5-methylfuran-2-yl)methylene)thiazolidin-4-one

The title compound was prepared in accordance with Examples 26 and 65. The product precipitated from the reaction mixture, was filtered off and recrystallised from acetic acid to give 139 mg of the title compound. LC-MS t_{R}: 1.6 min. m/z 319.2/321.2 (MH+). Major tautomer: ¹H NMR (400 MHz, CDCl₃ δ ppm: 2.38 (s, 3H), 6.20 (d, J=3.32 Hz, 1H), 6.73 (d, J=3.53 Hz, 1H), 7.42 (d, J=8.57 Hz, 2H), 7.17 (d, J=8.30 Hz, 2H), 7.52 (s, 1H) (total 10H). Minor tautomer (ca 20% vs. major): 2.47 (s, 0.64H), 6.25 (d, J=3.20 Hz, 0.20H), 6.82 (d, J=3.46 Hz, 0.20H), 7.24 (s, 0.29H), 7.49 (d, J=8.65 Hz, 0.46H), 7.66 (s, 0.18H) (total 1.97).

### Example 160

### 2-(4-Chlorophenylimino)-5-((5-methylfuran-2-yl)methyl)thiazolidin-4-one

A mixture of 2-(4-chlorophenylimino)-5-((5-methylfuran-2-yl)methylene)-thiazolidin-4-one (66.5 mg, 0.209 mmol; see Example 160) and sodium borohydride (26.5 mg, 0.701 mmol) in THF (0.8 mL) was heated in a closed screw-cap tube at 70° C. overnight. The reaction was quenched with methanol (1 mL) and acetic acid (1 mL), diluted with ethyl acetate and washed with water. The organic phase was dried with sodium sulfate, filtered and concentrated, and the crude product was purified by silica gel chromatography using petroleumether:ethyl acetate (2:1) as eluent to give 52 mg of the title compound. LC-MS (B) t_{R}: 1.5 min. m/z 321.3/323.2 (MH+). ¹H NMR: δCDCl₃): 8.26 (b, 1H), 7.33 (d, J=8.63 Hz, 2H), 7.12 (d, J=8.55 Hz, 2H), 5.97 (d, J=3.00 Hz, 1H), 5.85 (d, J=2.13 Hz, 1H), 4.42 (dd, J=10.41, 3.49 Hz, 1H), 3.54 (dd, J=15.37, 3.38 Hz, 1H), 3.02 (dd, J=15.46, 10.43 Hz, 1H), 2.22 (s, 3H).

### Example 161

### 2-(4-Chlorophenylimino)-5-((5-methylthiophen-2-yl)methylene)thiazolidin-4-one

The title compound was prepared in accordance with Examples 26 and 65. The product precipitated from the reaction mixture, was filtered off and recrystallised from acetic acid to give 106 mg of the title compound. LC-MS (B) t_{R}: 2.05 min. 335.85 (MH+).

### Example 162

### 2-(4-Chlorophenylimino)-5-((5-methylthiophen-2-yl)methyl)thiazolidin-4-one

A mixture of 2-(4-chlorophenylimino)-5-((5-methylthiophen-2-yl)methylene)-thiazolidin-4-one (33 mg, 0.0985 mmol; see Example 61) and sodium borohydride (13 mg, 0.343 mmol) in THF (0.8 mL) was refluxed overnight. The reaction was quenched with acetic acid (2 mL), diluted with ethyl acetate and washed with water. The organic phase was dried with sodium sulfate, filtered and concentrated, and the crude product was purified by silica gel column chromatography using petroleumether:ethyl acetate (2:1) as eluent to give 20 mg of the title compound as a yellow solid. LC-MS (B) t_{R}: 1.77 min. m/z 337 (MH+). ¹H NMR: δDMSO-d₆): 3.25 (s, 3H), 3.25 (ddd, 1H), 3.80 (ddd, 1H), 4.4 (dd, 1H), 4.56 (dd, 1H), 6.60 (d, 1H), 6.70 (d, 1H) tautomer, 7.20 (d, 2H), 7.50 (d, 2H).

### Example 163

### 5-(3-(Trifluoromethyl)benzyl)-2-(p-tolylimino)oxazolidin-4-one

A solution of ethyl 2-chloro-3-(3-(trifluoromethyl)phenyl)propanoate (610 mg, 2.17 mmol), p-methylphenylurea (337 mg, 2.25 mmol) and NaOAc (212 mg, 2.53 mmol) in 5.0 mL 95% EtOH was refluxed for 72 h and then concentrated. The residue was partitioned between EtOAc and water, and the water phase was extracted with EtOAc (3×). The combined organic phases were dried with MgSO₄, filtered and concentrated, and the crude product was purified by silica gel column chromatography using toluene:EtOAc 2:1 as eluent. Subsequent recrystallization from MeOH yielded 493 mg of the title compound as a white powder. LC-MS (A) t_{R}: 10.42 min. ES-MS m/z: 349.4 (MH+). ¹H NMR: δDMSO-d₆): 3.1 (s, 3H), 3.4 (m, 1H), 3.6 (m, 1H), 3.8 (m, 1H), 4.0 (m, 1H), 4.25-4.35 (ddd, 1H), 7.19 (m, 4H), 7.55 (m, 2H), 7.7 (m, 2H).

### Example 164

### [5-(3-Trifluoromethylbenzyl)-1,1-dioxo-1λ⁶-[1,4,2]dithiazolidin-3-ylidene]-(4-chloro)phenyl-2-amine

Sodium bis(trimethylsilyl)amide (0.6M, 1.06 mL, 0.63 mmol) was added dropwise to a solution of 1,1-dioxo-1λ⁶-[1,4,2]dithiazolidin-3-ylidene]-p-chlorophenyl-amine (33 mg, 0.12 mmol) in dry THF (2 mL) at −78° C. under nitrogen atmosphere. The reaction mixture was stirred at this temperature for 1 hour, before a solution of 3-trifluorobenzyl bromide (75 μL, 0.63 mmol) in dry THF (0.5 mL) was dropwise added. The temperature was kept at −78° C. for 5 h, and the reaction was quenched by addition of hydrochloric acid and EtOAc. The water phase was extracted with EtOAc (×3), and the combined organic phases were dried with Na₂SO₄, filtered and concentrated. The crude product was purified by silica gel δ(DMSO-d₆): 3.2 (dd, 1H), 3.6 (dd, 1H), 5.5 (dd, 1H), 7.4-7.5. (m, 2H), 7.6-7,7-. (m, column chromatography (toluene:EtOAc 100:0 to 2:1) to give 15 mg of the title compound. LC-MS (A) t_{R}: 10.89 min. ES-MS m/z: 421.2 (MH+). ¹H NMR: 4H), 7.7-7.8 (d, 1H), 7.8 (s, 1H).

### Example 165

### [5-(3-Trifluoromethylbenzyl)-1,1-dioxo-1λ⁶-[1,4,2]dithiazolidin-3-ylidene]-2-benzamide

The above compound is prepared in accordance with the procedures described herein.

### Example 166

### 5-(3-(Trifluoromethyl)benzyl)-4-methyl-N-(4-chlorophenyl)thiazol-2-amine

### (a) 3-Chloro-4-(3-(trifluoromethyl)phenyl)butan-2-one

A solution of sodium nitrite (0.31 g, 4.42 mmol) in water (0.9 ml) was added dropwise to a solution of 3-trifluoromethylaniline (0.50 ml, 4.02 mmol) in conc. hydrochloric acid (1.0 ml) and acetone (9.0 ml) under ice-water bath cooling. The mixture was stirred at 00° C. for 20 min. After addition of methyl vinyl ketone (2.00 ml, 24.11 mmol) and Cu₂O (26 mg) the mixture was stirred at 40° C. for 40 min. The reaction mixture was cooled to room temperature and poured into a sat. aq. NaHCO₃ solution. The water phase was extracted with CH₂Cl₂, the organic phase was dried over MgSO₄ and concentrated in vacuum to give a brown oil. The crude product was purified by silica gel chromatography using petroleum ether/EtOAc (0-5%) as eluent to give 605 mg of the title compound as a yellow oil. ¹H NMR: δ400 MHz) CDCl₃): 2.34 (s, 3H), 3.12 (dd, 1H), 3.41 (dd, 1H), 4.40 (m, 1H), 7.42-7.57 (m, 4H) ppm.

### (b) 5-(3-(Trifluoromethyl)benzyl)-4-methyl-N-(4-chlorophenyl)thiazol-2-amine

3-chloro-4-(3-(trifluoromethyl)phenyl)butan-2-one (200 mg, 0.80 mmol; see step (a) above), 4-chlorophenylthiourea (149 mg, 0.80 mmol) and NaOAc (72 mg, 0.88 mmol) were suspended in 95% EtOH (2 ml). The reaction mixture was refluxed for 72 h and the solvent was evaporated. The crude material was dissolved in EtOAc and extracted with water. The water phase was washed with EtOAc, and the organic phases were combined, dried with MgSO₄ and the solvent was evaporated. The crude product was purified by silica gel column chromatography using a gradient of petroleum ether/EtOAc (0-30%) as eluent and by recrystallisation from hot methanol yielding 157 mg of the title compound as white crystals. LC-MS (A) t_{R}: 10.68 min. ES-MS m/z 383.4 (MH+). ¹H NMR: δ400 MHz) DMSO-d₆): 2.19 (s, 3H), 4.08 (s, 2H), 7.29-7.31 (m; 2H), 7.50-7.61 (m, 6H) ppm.

### Biological Tests

### Test A

### Cell Proliferation Assay

### Reagents

Dulbecco's modified Eagle's medium (D-MEM)+1000 mg/L Glucose+GlutaMAX™1+Pyruvate (Gibco #21885-025)

### V/V Foetal Bovine Serum (Gibco 10500-064)

PEST (100 U/ml penicillin, 100 ug/ml streptomycin, Gibco 15140-122)
CyStain PI absolute T Kit (Partec #05-5023)
Linolenic acid 99%, L2376 from Sigma Aldrich
Dimethyl sulfoxide (DMSO)

### Equipment

Cytomics™ FC500 Flow Cytometer with CXP software (Beckman Coulter)
MDA-MB-231 cells

MDA-MB-231 cells were cultured in the propagation media D-MEM+1000 mg/L Glucose+GlutaMAX™1+Pyruvate supplemented with 10% V/V Foetal Bovine Serum and PEST (100 U/ml penicillin, 100 μg/mL streptomycin). Cells were seeded in 6 well plates to a density of 300 000 cells/well in propagation media. After 24 hours, media was replaced with serum free D-MEM media.

Linolenic acid was diluted in DMSO to a concentration of 100 mM and added to the culture media to a final concentration of 100 μM.

Compounds were as dissolved in DMSO to a concentrations of 10 mM (Compounds of Examples 95 and 6 (Compound X and Compound Y, respectively)) and 40 mM (Compound of Example 4 (Compound Z)) and added to the culture media to a final concentration of 10 μM (X and Y) and 40 μM (Z) respectively.

After 24 hours in serum free media DMEM, linolenic acid (to a final concentration of 10 μM) and compounds to be screened for activity were added to a final concentration of 10 μM (Compounds X and Y) and 40 μM (Compound Z) respectively. Final DMSO concentration was kept at 0.2% in all wells. After 24 hours of stimulation, cells were harvested and propidium iodine stained using a CyStain PI absolute T Kit according to manufacturer's recommendations. Cells were subsequently analyzed using a Cytomics™ FC500 Flow Cytometer with CXP software (Beckman Coulter) for cell cycle distribution. Cells were incubated with or without linolenic acid (LA) and the Compounds X, Y and Z for 24 hours at indicated concentrations. Cells in S-phase from untreated sample were set to 100% in each experiment.

### Results

The described method was shown to exhibit the sensitivity required to detect an antagonist to free fatty acid stimulation. The measurement of DNA synthesis for quantification of cell proliferation minimizes errors inherent in several other assays.

It was observed that FFA stimulation of MDA-MB-231 cells leads to an increased proliferation as demonstrated in FIGS. 1a and 1b, where the proportion of cells in S-phase of the cell cycle is increased in b versus a as measured by propidium iodine incorporation. This stimulatory effect of FFA could be attenuated by Compound X in a 10:1 molar ratio (FIG. 1c). These results indicate that Compound X is able to antagonize free fatty acid stimulated cell proliferation.

The experiment described was repeated 4 times and the results are summarized in FIG. 2A. Compounds Z and Y were also able to antagonize free fatty acid stimulated cell proliferation, as shown FIGS. 2B and 2C, respectively.

Thus, the relevant compounds attenuate the FFA induced cell proliferation in a human breast cancer cell line. The ability of Compounds X, Y and Z to inhibit such proliferation may be expressed as percentage antagonist activity as follows:

Compound X—70% at a concentration of 10 μM
Compound Y—100% at a concentration of 10 μM
Compound Z—50% at a concentration of 10 μM.

Similar experiments were conducted in respect of compounds of the examples above, which were also found to exhibit percentage antagonist activities at least 20% at a concentration of 10 μM.

### Test B

### In Vivo Mouse Model

week old Athymic BALB/cA nude mice were delivered from Taconic (Denmark) and kept under barrier conditions for 1 week acclimatisation. At 6 weeks, 17 mice were injected subcutaneously on the flank with 1.8×10⁶ MDA-MB-231 human breast cancer cells (LGC Promochem-ATCC) in a 50/50 v/v solution of phosphate buffered saline (PBS) (Gibco 10010-015, Invitrogen) Matrigel HC (BD Biosciences).

After 11 days, palpable tumors were observed in 16 mice. 2 mice were sacrificed and the tumors dissected and examined. 2 groups of 7 mice each were treated once daily by intraperitoneal injections of 1 mg/kg bodyweight of the compound of Example 6 (Compound Y) in PBS/1% v/v dimethylsufoxide or vehicle control respectively for 9 days. The mice were sacrificed by cervical dislocation and tumors were dissected.

### Histology

The tumor tissue were fixated overnight in PBS (containing 4% w/v paraformaldehyde (Scharlau PA0095, Sharlau Chemie SA, Spain) at +4° C. The tumor tissue were then cryopreserved by 24 hour incubation in PBS containing 30% w/v sucrose (BDH #102745C (www.vwr.com) at +4° C. and embedded in Tissue-Tek embedding media (Sakura Finetek Europa BV, Netherlands). 10 μm cryosections were generated an stained with Mayers Hematoxylin (Dako) for 5 min and destained for 3×10 minutes in tap water. Slides were mounted using Dako faramount aqueous mounting medium and examined using a Nikon Eclipse TS 100 microscope documented using a Nikon coolpix 4500.

### Results

The tumors from mice treated with test compound and vehicle were analyzed for morphology by microscopic examination of hematoxylin stained cryosections. The results are shown in FIGS. 3A to 3F.

FIG. 3A shows a hematoxylin stained section from a tumor dissected from a vehicle treated mouse at 10× magnification. It is to be noted that there is a relative abundance of cells in the interior of the section as well as the relative thickness of the uninterrupted zone of cell in the periphery of the section.

FIG. 3B shows a hematoxylin stained section from a tumor dissected from a vehicle treated mouse at 20× magnification. It is to be noted that the cells in the interior of the section display morphology consistent with adenocarcinoma.

FIG. 3C shows a hematoxylin stained section from a tumor dissected from a vehicle treated mouse at 40× magnification. It is to be noted that no cell displaying morphology indicative of macrophage/monocyte could be found.

FIG. 3D shows a hematoxylin stained section from a tumor dissected from a mouse treated with the Compound Y at 10× magnification. The low cell density in the interior of the section and the thin layer of cells displaying morphology is to be noted, which is consistent with poorly differentiated adenocarcinoma.

FIG. 3E shows a hematoxylin stained section from a tumor dissected from mouse treated with the Compound Y at 20× magnification. The lack of cells displaying fibroblast morphology in the interior of the section is to be noted.

FIG. 3F shows a hematoxylin stained section from a tumor dissected from a mouse treated with the compound of Compound Y at 40× magnification. The accumulation of cells displaying morphology indicative of macrophage/monocyte in the interior of the section (black arrows) is to be noted.

Thus, the main finding was thus that the cell-density in the interior of the tumors was markedly reduced in tumors dissected from test compound treated mice as compared to tumors from vehicle treated mice. Moreover, the majority of the cells found in the interior of the sections from the treated group displayed a morphology inconsistent with adenocarcinoma while cells displaying macrophage/monocyte morphology was a frequent finding. In contrast, only one of seven tumors from the vehicle treated group showed indication of macrophage/monocyte infiltration.

In summary, these findings show a correlation between treatment with test compound and reduction of cancer cells in the xenograft tumors.

1.-31. (canceled)

32. A method of treating cancer which comprises administering, to a subject in need of such treatment, an effective amount of a compound of formula I, 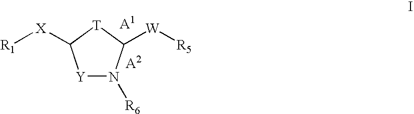
wherein
X represents —[C(R₈)(R₉)]ₙ—;
n represents 0, 1, 2 or 3;
Y represents —C(O)—, —S(O)₂— or ═C(R₁₀)—;
T represents —S— or —O—;
W represents —NR₇—, —CR₇R₇—, —NR₇C(O)—, —NR₇S(O)₂—, —NR₇C(O)NR₇—, —NR₇C(O)O— or a bond;
one of A₁ or A₂ represents a double bond and the other represents a single bond;
when A₁ represents a single bond, A₂ is a double bond and R₆ is absent;
when A₂ represents a single bond, A₁ is a double bond and, if present, one R₇ (which is attached α to the requisite ring of the compound of formula I) is absent;
R₁ represents —C(O)NR₃R₂, —NR₃R₂, —C(O)OR₂, —NR₄C(O)NR₃R₂, —NR₄C(O)OR₂, —OC(O)NR₃R₂, —NR₄C(O)R₂, —OC(O)R₂, —OR₂, —SR₂, H, alkyl, cycloalkyl, heterocyclyl, benzyl, aryl or heteroaryl (which latter six groups are optionally substituted by one or more groups selected from B¹, B², B³, B⁴, B⁵ and B⁶, respectively);
R₂ and R₅ independently represent hydrogen, alkyl, cycloalkyl, heterocyclyl, benzyl, aryl or heteroaryl (which latter six groups are optionally substituted by one or more groups selected from B⁷, B⁸, B⁹, B¹⁰, B¹¹ and B¹², respectively);
R₃, R₄, R₆ and R₇ independently represent hydrogen, alkyl, cycloalkyl, aryl or benzyl (which latter four groups are optionally substituted by one or more groups selected from B¹³, B¹⁴, B¹⁵ and B¹⁶, respectively), or heterocyclyl or heteroaryl (which latter two groups are optionally substituted by one or more groups selected from B¹⁴ and B¹⁵, respectively);
R₈ and R₉ are independently selected from hydrogen, alkyl and aryl (which latter two groups are optionally substituted by B^{16a} and B^{16b}, respectively);
R₁₀ represents hydrogen, alkyl or aryl (which latter two groups are optionally substituted by one or more groups selected from B¹⁷ and B¹⁸, respectively);
B¹ to B¹⁸ independently represent cyano, —NO₂, halo, —OR₁₁, —NR₁₂R₁₃, —SR₁₄,
—Si(R₁₅)₃, —C(O)OR₁₆, —C(O)NR₁₆ₐR_{16b}, —S(O)₂NR_{16c}R_{16d}, aryl or heteroaryl (which aryl and heteroaryl groups are themselves optionally and independently substituted by one or more groups selected from halo and R₁₇); or, alternatively,
B⁴, B⁵, B⁶, B¹⁰, B¹¹, B¹², B¹⁵, B¹⁶, B^{16b} or B¹⁸ independently represent R₁₇;
R₁₁, R₁₂, R₁₃, R₁₄, R₁₆, R₁₆ₐ, R_{16b}, R_{16c} and R_{16d} independently represent H or R₁₇; and
R₁₅ and R₁₇ independently represent C₁₋₆ alkyl optionally substituted by one or more halo atoms,
or a pharmaceutically-acceptable salt or solvate, or a pharmaceutically functional derivative thereof,
for the manufacture of a medicament for the treatment of cancer,
provided that, when n represents 0 and R₁ represents an optionally substituted alkyl group, then that alkyl group is saturated.

33. A method as claimed in claim 32 wherein, in the compound of formula I, T represents —S—,

34. A method as claimed in claim 32 wherein, in the compound of formula I, Y represents —C(O)—.

35. A method as claimed in claim 32 wherein, in the compound of formula I, R₁₀ represents H or alkyl.

36. A method as claimed in claim 32 wherein, in the compound of formula I, W represents —NR₇—, —NR₇C(O)—, —NR₇C(O)O—, —NR₇C(O)NR₇— or —NR₇S(O)₂—.

37. A method as claimed in claim 32 wherein, in the compound of formula I, R₅ represents optionally substituted C₁₋₃ alkyl, cycloalkyl or optionally substituted phenyl or optionally substituted heteroaryl.

38. A method as claimed in claim 32 wherein, in the compound of formula I, n represents 1, 2 or 3.

39. A method as claimed claim 32 wherein, in the compound of formula I, R₈ and R₉ independently represent C₁₋₃ alkyl or H.

40. A method as claimed in claim 32 wherein, in the compound of formula I, R₁ represents alkyl, —NR₃R₂, —OR₂, —SR₂, —NR₄C(O)R₂, —NR₄C(O)NR₃R₂, —NR₄C(O)OR₂, —C(O)NR₃R₂, —C(O)OR₂, optionally substituted heteroaryl or optionally substituted phenyl.

41. A method as claimed in claim 40 wherein R₁ represents optionally substituted furanyl, thienyl or phenyl.

42. A method as claimed in claim 32 wherein, in the compound of formula I, R₄ or R₃ independently represent C₁₋₃ alkyl or H.

43. A method as claimed in claim 32 wherein, in the compound of formula I, R₂ represents optionally substituted C₁₋₃ alkyl, optionally substituted phenyl or H;

44. A method as claimed in claim 32 wherein, in the compound of formula I, when W represents —NR₇— and R₇ is absent, then R₆ represents H, C₁₋₆ alkyl or phenyl, which latter two groups may be substitutued by one or more of B¹³ and B¹⁵, respectively.

45. A method as claimed in claim 32 wherein, in the compound of formula I, when W represents —NR₇— and R₆ is absent, then R₇ represents C₁₋₃ alkyl, phenyl or benzyl, all of which may be substituted by one or more B¹³, B¹⁵ and B¹⁶, respectively.

46. A method as claimed in claim 32 wherein, in the compound of formula I, when W represents —CR₇R₇—, then A₂ represents a double bond.

47. A method as claimed in claim 32 wherein, in the compound of formula I, when W represents —CR₇R₇—, then each R₇ independently represents C₁₋₃ alkyl or H.

48. A method as claimed in claim 32 wherein, in the compound of formula I, B¹ to B¹⁸ independently represent cyano, NO₂, halo, —OR₁₁, —C(O)OR₁₆, —C(O)NR₁₆ₐR_{16b} or —S(O)₂NR_{6c}R_{16d}; and/or B⁴ to B⁶, B¹⁰ to B¹², B¹⁵, B¹⁶ and B¹⁸ independently represent R₁₇; and/or B¹ to B¹⁸ independently represent heteroaryl or phenyl, both of which may be substituted by one or more groups selected from halo or R₁₇.

49. A method as claimed in claim 32 wherein, in the compound of formula I, R₁₁ represents C₁₋₃ alkyl or H.

50. A method as claimed in claim 32 wherein, in the compound of formula I, R₁₆ represents H or C₁₋₃ alkyl.

51. A method as claimed in claim 32 wherein, in the compound of formula I, R₁₆ₐ, R_{16b}, R_{16c} and R_{16d} independently represent C₁₋₂ alkyl or H.

52. A method as claimed in claim 32 wherein, in the compound of formula I, R₁₇ represents C₁₋₄ alkyl optionally substituted by one or more halo atoms.

53. A method as claimed in claim 32 where the compound of formula I is selected from:
5-(4-fluorobenzyl)-2-(pyridin-2-ylimino)thiazolidin-4-one;
5-(p-methylbenzyl)-2-(4-chlorophenylimino)thiazolidin-4-one;
5-(3-(trifluoromethyl)benzyl)-2-(p-tolylimino)thiazolidin-4-one;
5-(3-(trifluoromethyl)benzyl)-2-(4-chlorophenylimino)thiazolidin-4-one;
5-(3-(trifluoromethyl)benzyl)-2-(4-isopropylphenylimino)thiazolidin-4-one;
5-(3-(trifluoromethyl)benzyl)-2-(4-methoxyphenylimino)thiazolidin-4-one;
5-(3-(trifluoromethyl)benzyl)-2-(phenylimino)thiazolidin-4-one;
2-(3,4-dichlorophenylimino)-5-(3-(trifluoromethyl)benzyl)thiazolidin-4-one;
2-(2,4-dichlorophenylimino)-5-(3-(trifluoromethyl)benzyl)thiazolidin-4-one;
5-(3-(trifluoromethyl)benzyl)-2-(p-tolylimino)-3-methylthiazolidin-4-one;
N-(5-(3-(trifluoromethyl)benzyl)-4-oxothiazolidin-2-ylidene)-4-chlorobenzamide;
5-(3-(trifluoromethyl)benzyl)-2-(4-chlorophenyl)sulfonyliminothiazolidin-4-one; phenyl 5-(3-(trifluoromethyl)benzyl)-4-oxothiazolidin-2-ylidenecarbamate;
5-(4-methoxyphenethyl)-2-(p-tolylimino)thiazolidin-4-one;
5-(4-methoxyphenethyl)-2-(phenylimino)thiazolidin-4-one; and
2-(p-tolylimino)-5-phenethylthiazolidin-4-one.

54. A method as claimed in claim 53 wherein the compound is 5-(3-(trifluoromethyl)benzyl)-2-(4-chlorophenylimino)thiazolidin-4-one.

55. A method as claimed in claim 32 wherein the cancer is of the colon, the breast or the prostate.

56. A compound of formula I as defined in claim 32 but in which Y represents —S(O)₂—, or a pharmaceutically-acceptable salt or solvate, or a pharmaceutically functional derivative thereof, provided that when T represents —S—, W represents —NR₇— and:
(a) A₁ represents a double bond, n represents 0 and R₁ represents phenyl, then (i) R₅ does not represent phenyl when R₆ represents methyl and (ii) R₆ does not represent phenyl when R₅ represent methyl; and
(b) A₂ represents a double bond, n represents 1, R₁, R₇, R₈ and R₉ all represent H, then R₅ does not represent 3-chlorobenzyl.

57. A compound of formula I as defined in claim 32 wherein n represents 1 or 2.

58. A compound as defined in claim 32 or as claimed in claim 57 wherein R₈ and R₉ both represent H.

59. A compound as defined in claim 32 or as claimed in claim 57 wherein R₁ represents aryl optionally substituted by B⁵.

60. A compound as claimed in claim 59 wherein R₁ represents phenyl substituted by B⁵.

61. A compound as defined in claim 32 or as claimed in claim 57 wherein B⁵ represents R₁₇.

62. A compound as defined in claim 32 or as claimed in claim 57 wherein R₁₇ represents C₁₋₄ alkyl optionally substituted by one or more halo atoms.

63. A compound as claimed in claim 62 wherein R₁₇ represents C₁₋₃ alkyl substituted by one or more halo atoms.

64. A compound as claimed in claim 63 wherein R₁₇ represents C₁₋₃ alkyl substituted by one or more fluoro atoms.

65. A compound as claimed in claim 64 wherein R₁₇ represents methyl substituted by one or more fluoro atoms.

66. A compound as defined in claim 32 or as claimed in claim 57 wherein T represents S.

67. A compound as defined in claim 32 or as claimed in claim 57 wherein R₆ represents H.

68. A compound as defined in claim 56, or a pharmaceutically-acceptable salt or solvate, or a pharmaceutically functional derivative thereof, for use as a pharmaceutical.

69. A pharmaceutical formulation including a compound as defined in claim 56, or a pharmaceutically-acceptable salt or solvate, or a pharmaceutically functional derivative thereof, in admixture with a pharmaceutically acceptable adjuvant, diluent or carrier.

70. A combination product comprising:
(A) a compound of formula I as defined in claim 32, or a pharmaceutically-acceptable salt or solvate, or a pharmaceutically functional derivative thereof; and
(B) another therapeutic agent useful in the treatment of cancer,
wherein each of components (A) and (B) is formulated in admixture with a pharmaceutically-acceptable adjuvant, diluent or carrier.

71. A combination product as claimed in claim 70 which comprises a pharmaceutical formulation including a compound of formula I as defined in claim 32, or a pharmaceutically-acceptable salt or solvate, or a pharmaceutically functional derivative thereof; another therapeutic agent useful in the treatment of cancer; and a pharmaceutically-acceptable adjuvant, diluent or carrier.

72. A combination product as claimed in claim 70, which comprises a kit of parts comprising components:
(a) a pharmaceutical formulation including a compound of formula I as defined in claim 32, or a pharmaceutically-acceptable salt or solvate, or a pharmaceutically functional derivative thereof, in admixture with a pharmaceutically-acceptable adjuvant, diluent or carrier; and
(b) a pharmaceutical formulation including another therapeutic agent useful in the treatment of cancer in admixture with a pharmaceutically-acceptable adjuvant, diluent or carrier,
which components (a) and (b) are each provided in a form that is suitable for administration in conjunction with the other.
